(19) United States
(12) Patent Application Publication
(10) Pub. No.: US 2013/0102876 A1
(43) Pub. Date: Apr. 25, 2013

(54) Port System and Methods

(71) Applicants: Timothy A. Limon, Cupertino, CA (US); Brian K. Shiu, Sunnyvale, CA (US); Theodore M. Bender, San Anselmo, CA (US); Joshua Makower, Los Altos, CA (US)

(72) Inventors: Timothy A. Limon, Cupertino, CA (US); Brian K. Shiu, Sunnyvale, CA (US); Theodore M. Bender, San Anselmo, CA (US); Joshua Makower, Los Altos, CA (US)

(21) Appl. No.: 13/707,580

(22) Filed: Dec. 6, 2012

Related U.S. Application Data
(63) Continuation-in-part of application No. 13/015,086, filed on Jan. 27, 2011.
Application No. 13/015,086 is a continuation-in-part of application No. 12/474,226, filed on May 28, 2009.
Application No. 12/474,226 is a continuation-in-part of application No. 11/716,985, filed on Mar. 10, 2007, now Pat. No. 8,342,183.
Application No. 11/716,985 is a continuation-in-part of application No. 11/716,986, filed on Mar. 10, 2007.
Application No. 11/716,986 is a continuation-in-part of application No. 12/473,818, filed on May 28, 2009.
Application No. 12/473,818 is a continuation-in-part of application No. 11/716,985, filed on Mar. 10, 2007, now Pat. No. 8,342,183.
Application No. 11/716,985 is a continuation-in-part of application No. 11/716,986, filed on Mar. 10, 2007.
Application No. 11/716,986 is a continuation-in-part of application No. 12/474,118, filed on May 28, 2009.
Application No. 12/474,118 is a continuation-in-part of application No. 11/716,985, filed on Mar. 10, 2007, now Pat. No. 8,342,183.
Application No. 11/716,985 is a continuation-in-part of application No. 11/716,986, filed on Mar. 10, 2007.
Application No. 11/716,985 is a continuation-in-part of application No. 11/407,701, filed on Apr. 19, 2006, now Pat. No. 8,070,768.
Application No. 13/015,086 is a continuation-in-part of application No. 11/407,701, filed on Apr. 19, 2006, now Pat. No. 8,070,768.
Application No. 11/407,701 is a continuation-in-part of application No. 11/974,444, filed on Oct. 11, 2007.
(60) Provisional application No. 61/568,617, filed on Dec. 8, 2011.

(51) Int. Cl. A61F 5/00; A61B 5/05; A61B 1/018; A61M 5/00; A61M 25/10

(52) U.S. Cl. CPC A61F 5/0056; A61M 5/00; A61M 25/10; A61B 1/018; A61B 5/05
USPC 600/409; 604/175; 604/96.01; 600/37; 600/106; 604/99.01

(57) ABSTRACT

An implantable port system which in at least one embodiment includes a base having an upper surface configured to be positioned under and in contact with a first tissue surface in a body, and a port housing having a lower surface configured to be positioned over a second tissue surface above the first tissue surface. The base and the port housing may be separate from one another and do not contact each other at the time of positioning the base. The base and port may be connected together upon positioning the port in contact with the second tissue surface. A method of implanting a port system may include installing at least a portion of a base of the system beneath a tissue layer in a body, and attaching a port housing to the base at a location above the tissue layer, wherein the port housing is not in contact with the base during the installation of a least a part of the base system beneath the tissue layer.

### CROSS-REFERENCE

This application is a continuation-in-part application of co-pending application Ser. No. 13/015,086 filed Jan. 27, 2011 and titled Minimally Invasive, Direct Delivery Methods for Implanting Obesity Treatment Devices; which is a continuation-in-part application of co-pending application Ser. No. 12/474,226, filed May 28, 2009, which is a continuation-in-part application of application Ser. No. 11/716,985, filed Mar. 10, 2007 and a continuation-in-part application of application Ser. No. 11/716,986, filed Mar. 10, 2007, and we hereby claim priority to each of the foregoing applications under 35 USC §120. Each of the foregoing applications is also hereby incorporated herein, in its entirety, by reference thereto.

This application is a continuation-in-part application of co-pending application Ser. No. 12/473,818, filed May 28, 2009, which is a continuation-in-part application of application Ser. No. 11/716,985, filed Mar. 10, 2007 and a continuation-in-part application of application Ser. No. 11/716,986, filed Mar. 10, 2007, and we hereby claim priority to each of the foregoing applications under 35 USC §120. Each of the foregoing applications is also hereby incorporated herein, in its entirety, by reference thereto.

This application is a continuation-in-part application of co-pending application Ser. No. 12/474,118, filed May 28, 2009, which is a continuation-in-part application of application Ser. No. 11/716,985, filed Mar. 10, 2007 and a continuation-in-part application of application Ser. No. 11/716,986, filed Mar. 10, 2007, and we hereby claim priority to each of the foregoing applications under 35 USC §120. Each of the foregoing applications is also hereby incorporated herein, in its entirety, by reference thereto.

This application is a continuation-in-part application of co-pending application Ser. No. 11/407,701, filed Apr. 19, 2006 to which application we claim priority under 35 USC §120 and which application is incorporated herein, in its entirety, by reference thereto.

Both application Ser. Nos. 11/716,985 and 11/716,986 are continuation-in-part applications of application Ser. No. 11/407,701, filed Apr. 19, 2006, now U.S. Pat. No. 8,070,768 which issued on Dec. 6, 2011, to which application and patent we claim priority under 35 USC §120 and which application and patent are incorporated herein, in their entireties, by reference thereto.

This application claims the benefit of U.S. Provisional Application No. 61/568,617, filed Dec. 8, 2011 and titled “Port Systems and Methods”, which application is hereby incorporated herein, in its entirety, by reference thereto

### FIELD OF THE INVENTION

The present invention relates generally to medical implants, and more particularly to attachment mechanisms for use with a variety of medical implants, to attach the medical implants to body tissue.

### BACKGROUND OF THE INVENTION

There are many examples of medical implants currently in use, including, but not limited to injection ports (such as used with inflatable implants used to treat obesity), vascular access ports, cardiac pacemaker devices, gastric pacing devices. Such implants are typically attached subcutaneously, in a location where they can be easily accessed and where they can function.

A traditional surgical technique for securing a fluid injection port such as a vascular access port or an injection port used for an obesity treatment implant such as an inflatable implant (extra-gastric or intra-gastric or gastric band) involves applying sutures through a series of holes spaced circumferentially about a base flange of the port to stitch the port to the tissue. This technique can be time consuming and may be difficult, particularly when performed on a morbidly obese patient, as fat tissue may obstruct the procedure and make it difficult to throw the sutures through, so as to reach the tissue layer intended to be attached to, as fat tissue is inadequate for attachment purposes.

Problems present with currently available injection ports and techniques for attaching them include port dislodgement or inversion, see Piorkowski et al., “Preventing port site inversion in laparoscopic adjustable gastric banding”, American Society for Bariatric Surgery, 3 (2007) 159-162, which is hereby incorporated herein, in its entirety, by reference thereto.

There is a continuing need for attachment mechanisms used to attach medical implants to body tissue, that reduce the risks of the medical implant attached thereby from rotating or inverting.

There is a continuing need for attachment mechanisms used to attach medical implants to body tissue, wherein the time required to perform the attachment of the attachment mechanism is significantly reduced relative t currently existing mechanisms and techniques.

There is a continuing need for attachment mechanisms used to attach medical implants to body tissue, wherein the mechanism maintains the location of the medical implant securely in place.

There is a continuing need for attachment mechanisms used to attach medical implants to body tissue, wherein the mechanism is operable to quickly detach the implant from the tissue.

There is a continuing need for attachment mechanisms used to attach medical implants to body tissue, wherein the mechanism is operable to reattach the implant, such as when the implant has been detached and repositioned.

The present invention meets all of the above needs and more.

### SUMMARY OF THE INVENTION

Port systems, methods and instruments for installing port systems are disclosed.

In one aspect of the present invention, an implantable port system is provided, including: a base having an upper surface configured to be positioned under and in contact with a first tissue surface in a body; and a port housing having a lower surface configured to be positioned over a second tissue surface above the first tissue surface; wherein the base and the port housing are separate from one another and do not contact each other at the time of positioning the base; and wherein the base and the port are connected together upon positioning the port in contact with the second tissue surface.

In at least one embodiment, the system includes a tissue-ingrowth encouraging layer on a top side of the base.

In at least one embodiment, the system includes a tissue-ingrowth encouraging layer on a bottom side of the port housing.

In at least one embodiment, a bottom surface of the base comprises a tissue-ingrowth discouraging layer to discourage adhesions and tissue ingrowth.

In at least one embodiment, the system includes means for quickly connecting the port housing to the base, the means for quickly connecting further being operable to quickly disconnect the port housing from the base.

In at least one embodiment, the system includes a base comprising a first base portion, the upper surface being provided on the first base portion, the base further comprising a second base portion have a lower surface, the lower surface of the second base portion configured to be positioned over and in contact with the second tissue surface, wherein the first and second base portions sandwich tissue including portions of the first and second tissue surfaces therebetween.

In at least one embodiment, the system includes a quick release mechanism operable to mechanically connect the port housing to the base and further being operable to quickly disconnect the port housing from the base.

In at least one embodiment, the system includes a ribbon interconnecting the first and second base portions, the ribbon being passable through the tissue layer.

In at least one embodiment, the base is inflatable.

In at least one embodiment, the port housing comprises a first plurality of protrusions and the base comprises a second plurality of receptacles, wherein the first plurality is a smaller number than the second plurality, whereby the port housing can be connected to the base at different relative locations through different sets of the receptacles.

In at least one embodiment, the protrusions are variably adjustable in the receptacles to vary a distance between the port housing and the base to accommodate various thicknesses of tissue layers.

In at least one embodiment, the second base portion comprises a first plurality of protrusions and the first base portion comprises a second plurality of receptacles, wherein the first plurality is a smaller number than the second plurality, whereby the second base portion can be connected to the first base portion at different relative locations through different sets of the receptacles.

In at least one embodiment, the system further includes a feature configured to facilitate location of the port housing when implanted subcutaneously.

In at least one embodiment, the feature comprises a foam layer.

In another aspect of the present invention, an implantable port system includes: base means for attachment to and contact with a tissue layer in a body; and port means for connection to the base means after attachment of the base means to the tissue layer, wherein the port means are detachable from the base means after connecting the port means and the base means together, such that the port means are quickly releasable from and quickly attachable to the base means.

In at least one embodiment, the base means comprises a lower base portion configured and dimensioned to be attached beneath an abdominal muscle tissue layer, and an upper base portion attachable to an upper surface of the abdominal muscle tissue layer or above the upper surface of the abdominal muscle tissue layer.

In at least one embodiment, the port means is connectable to and disconnectable from the base means via a mechanical, quick-release mechanism.

In at least one embodiment, the base means comprises a lower base portion configured and dimensioned to be attached beneath the tissue layer and an upper base portion configured and dimensioned to be attached above the tissue layer, the base means further comprising mechanical connectors configured to pass through the tissue layer and connect the upper and lower base portions together.

In at least one embodiment, the base means is inflatable.

In another aspect of the present invention, a port implantation system includes: a first elongate tube having a blunt distal end, the first elongate tube having a lumen dimensioned to receive an endoscope therein and at least a portion of the blunt distal end being transparent to allow viewing through the at least a portion thereof, the first elongate tube comprising a first mount temporarily connectable to a lower base portion of an implantable port system to mount the lower base portion thereto, at a location proximal of the blunt distal end; and a second elongate tube having an open distal end and a lumen dimensioned to receive the first elongate tube therein, the second elongate tube comprising a second mount temporarily connectable to an upper base portion of the implantable port system to mount the upper base portion thereto, such that, when the port implantation system is assembled, the upper base portion is proximal of the lower base portion.

In at least one embodiment, the blunt distal end comprises an opening therethrough to allow passage of a tip of the endoscope.

In at least one embodiment, the blunt distal end comprises a window for viewing therethrough.

In at least one embodiment, the system includes a third elongate tube dimensioned to be received in the second elongate tube, wherein a distal end portion of the third elongate tube comprises a housing mount temporarily connectable to a port housing of the implantable port system to mount the port housing thereto, and to advance the port housing through the second elongate tube and attach the port housing to the base after connecting the lower base portion to the upper base portion.

In at least one embodiment, the system is configured to remove the first elongate tube from the second elongate tube before inserting the third elongate tube into the second elongate tube.

In at least one embodiment, the system includes an endoscope inserted in the first elongate tube.

In at least one embodiment, the first elongate tube is inserted in the second elongate tube; the lower base portion is mounted on the first elongate tube, proximal of the blunt distal end; and the upper base portion is mounted on the second elongate tube proximal of the lower base portion.

In at least one embodiment, the system includes attachment members extending proximally from the lower base portion.

In another aspect of the present invention, a method of implanting a port system is provided, including: installing at least a portion of a base of the system beneath a tissue layer in a body; and attaching a port housing to the base at a location above the tissue layer, wherein the port housing is not in contact with the base during the installing a least a portion of the base system beneath the tissue layer.

In at least one embodiment, the base is inflatable.

In at least one embodiment, the tissue layer is fascia.

In at least one embodiment, the tissue layer comprises abdominal muscle.

In at least one embodiment, the method includes, following insertion of an expandable member having a fill tube extending proximally therefrom, the base is installed over the fill tube and at least partially through an opening through which the expandable member was inserted.

In at least one embodiment, the installing includes visualizing, using an endoscope, at least placement of at least a portion of the base beneath the tissue layer.

In at least one embodiment, the installing comprises delivering the base through an elongate tube and expanding the base to have an outside diameter larger than an inside diameter of the elongate tube; and the attachment of the port housing comprises manipulating attachment features to interconnect the base and the port, and cinching the port housing against one of the base and the tissue layer.

In at least one embodiment, the base comprises a balloon and the balloon is anchored beneath the tissue layer, the method further including attaching a platform above the tissue layer, to the base; and wherein the attaching a port housing to the base comprises docking the port housing to the platform.

In at least one embodiment, the port housing is attached to the platform by a mechanism, which, in a first configuration, positions the port housing in a relatively recessed position relative to the platform, and when in a second configuration, the port housing extends upwardly from the platform.

In at least one embodiment, the method includes pressing on and releasing pressure from the port housing to change from the first configuration to the second configuration, and from the second configuration to the first configuration, wherein the second configuration facilitates locating the port housing by touch, when use for inflation adjustment is desired.

In at least one embodiment, the mechanism is a magnetically driven mechanism, the method further comprising applying a magnetic field to the mechanism to change from the first configuration to the second configuration, and from the second configuration to the first configuration, wherein the second configuration facilitates locating the port housing by touch, when use for inflation adjustment is desired.

In at least one embodiment, the installing comprises inserting the base beneath the tissue layer; and passing tethers through the base and the tissue layer, wherein the passing is in a motion from a distal to a proximal direction.

In at least one embodiment, the attaching of the port housing comprises passing the tethers through the port housing in a distal to proximal direction; passing the port housing over the tethers to a position in contact with the tissue layer; and cinching the base and port housing together, thereby fixing the relative positions of the base and port housing on the tethers.

In at least one embodiment, the base is delivered beneath the tissue layer through an elongate tube.

In at least one embodiment, the method further includes removing the elongate tube after passing the tethers through the base and the tissue layer.

In at least one embodiment, the at least a portion comprises a lower base portion, and the installing comprises inserting a blunt tip of an instrument and the lower base portion mounted proximally of the blunt tip through an opening in the abdominal muscle; passing connectors proximally from the lower base portion, through the tissue layer and connecting to an upper base portion of the base.

In at least one embodiment, the attaching a port housing comprises: attaching the port housing to a tube passing through the base; and attaching the port housing to the upper base portion.

In at least one embodiment, the blunt tip is a portion of a first elongate tube of an instrument upon which the lower base potion is mounted; wherein the instrument further comprises a second elongate tube having a central opening through which the first elongate tube is inserted; and wherein the upper base portion is mounted to a distal end portion of the second elongate tube, wherein the installing comprises: passing the first and second elongate tubes through an opening in the abdominal muscle; displacing bowel by advancing the blunt tip thereagainst; retracting the second elongate tube from the opening in the abdominal muscle, thereby allowing the abdominal muscle and fascia to impinge upon the first elongate tube.

In at least one embodiment, the passing connectors comprises retracting the first elongate tube while substantially maintaining a position of the second elongate tube.

In at least one embodiment, the method includes visualizing at least one step of the method, using an endoscope inserted into the first elongate tube.

In at least one embodiment, the attaching the port housing comprises screwing the port housing into the base, wherein the port housing comprises a first set of threads and the base comprises a second set of threads that mate with the first set of threads.

In at least one embodiment, the attaching the port housing comprises snapping the port housing into the base.

In at least one embodiment, the installing at least a portion of a base comprises inserting an anvil of an instrument and the lower base portion mounted proximally of the anvil through an opening in the abdominal muscle; and passing connectors distally through the abdominal muscle and the lower base portion, wherein the anvil prevents penetration of bowel during the passing.

In at least one embodiment, the connectors comprise T-bars.

In at least one embodiment, the at least a portion comprises a lower base portion; wherein the lower base portion is fixed to an expandable member and the port system is configured to expand the expandable member upon injection of fluid through the port system; wherein the installing comprises: inserting the expandable member and lower base portion through an opening in the abdominal muscle; and passing connectors proximally from the lower base portion, through the abdominal muscle.

In at least one embodiment, the method includes connecting the connectors to an upper base portion of the base above the tissue layer; and wherein the attaching a port housing comprises: attaching the port housing to a tube passing through the base; and attaching the port housing to the upper base portion.

In at least one embodiment, the attaching a port housing comprises: attaching the port housing to a tube passing through the base; and attaching the connectors to the port housing.

In at least one embodiment, the method includes attaching the connectors to one of the port housing or an upper base portion of the base, the method further comprising connecting a ribbon between the lower base portion and one of the port housing and the upper base portion, wherein the ribbon passes through the opening and is configured to encourage tissue ingrowth therein.

In at least one embodiment, the at least a portion comprises a lower base portion; wherein the lower base portion is fixed to an expandable member and the port system is configured to expand the expandable member upon injection of fluid through the port system; wherein the installing comprises: inserting the expandable member and lower base portion through an opening in the abdominal muscle; and passing connectors distally from an upper base portion, located above the abdominal muscle into the lower base portion, thereby connecting the upper base portion to the lower base portion.

In another aspect of the present invention, a method of implanting a port system into a body includes: engaging tissue peripherally adjacent an opening in a tissue layer; positioning a base over the opening, positioning a port housing over the base; and connecting the base and the port housing together and to the tissue layer while drawing the engaged tissue closer to a center of the opening.

In at least one embodiment, a tube passes through the opening and the base, the method including connecting the port housing to the tube prior to the connecting the base and the port housing together.

In at least one embodiment, the base comprises a set of outer openings and a set of inner openings, the inner openings located radially inwardly of corresponding ones of the outer openings, wherein the engaging tissue comprises penetrating the tissue via penetrating instruments inserted through the outer openings.

In at least one embodiment, the method includes flexible tethers, each connected at a distal end to an anchor releasably mounted to one of the penetrating instruments, the flexible tethers further being routed through respective ones of the inner opening and through openings in the port housing, wherein the connecting comprises tensioning the flexible tethers, from proximal end locations proximal of the port housing, cinching the port housing and the base down against the tissue layer and together, while the engaged tissues are also drawn radially inwardly, as directed by drawing against the inner openings.

In at least one embodiment, a tube passes through the opening and the base, the method including connecting the port housing to the tube prior to the connecting the base and the port housing together, and wherein the engaged tissues are drawn up to the tube during the connecting.

In another aspect of the present invention, an implantable port system is provided that includes: a base having a surface dimensioned to be attached over an opening through a tissue layer in a body; and a port housing and a connector useable to connect the port housing to the base; wherein the base comprises a set of outer openings and a set of inner openings, the inner openings located radially inwardly of corresponding ones of the outer openings.

In at least one embodiment, the system further includes a set of grooves, each one of the set of grooves extending radially between corresponding ones of the inner and outer openings.

In at least one embodiment, the system further includes penetrating instruments extendable through the outer openings and flexible tethers extendable through the inner openings, the flexible tethers being releasably mountable, at distal end portions thereof, to respective ones of distal end portions of the penetrating instruments.

In at least one embodiment, the system further includes anchors fixed to distal ends of the flexible tethers, the anchors configured to be releasably mounted to the distal end portions of the penetrating instruments.

In another aspect of the present invention, an implantable port system is provided, including a lower base having an upper surface configured to be positioned under an opening through tissue and in contact with a first tissue surface in a body; an upper base having a lower surface configured to be positioned over the opening and in contact with a second tissue surface in the body; and an injection port housing having a lower surface configured to be attached to the upper base; wherein the lower base and the upper base each has a span dimension than a span dimension of the opening; and wherein the lower base is compressible to a compressed configuration having a compressed span dimension less than the span dimension of the opening that allows the lower base to be passed through the opening; and the lower base is resilient, wherein, upon passing the lower base through the opening and removing compressive forces from the lower base, the lower base resiliently returns to the span dimension greater than the span dimension of the opening, thereby preventing the lower base from passing back through the opening.

In another aspect of the present invention, a method of implanting a port device is provided, the method including: accessing an incision in a patient having been previously used to deliver an implant device therethrough; subcutaneously tunneling, through subcutaneous fat, away from the incision to a port target implant location; and subcutaneously attaching the port device to the patient at the port target implant location.

In another aspect of the present invention, an instrument for implanting a port device is provided, the system including: means for accessing an incision in a patient having been previously used to deliver an implant device therethrough; means for subcutaneously tunneling, through subcutaneous fat, away from the incision to a port target implant location; and means for subcutaneously attaching the port device to the patient at the port target implant location.

These and other features of the invention will become apparent to those persons skilled in the art upon reading the details of the systems, methods and instruments as more fully described below.

### BRIEF DESCRIPTION OF THE DRAWINGS

FIGS. 1A-1G illustrate various stages in a method of attaching an implantable port system to a layer of material according to an embodiment of the present invention.

FIG. 2A illustrates a port-base deployment tool according to an embodiment of the present invention.

FIG. 2B illustrates a distal end portion of the instrument of FIG. 2B.

FIG. 2C is a longitudinal sectional view of the distal end portion of FIG. 2A inserted into an abdominal cavity, according to an embodiment of the present invention.

FIG. 2D illustrates the abdominal wall surrounding an exposed tube being allowed to elastically return towards its undeformed conformation, according to an embodiment of the present invention.

FIGS. 2E-2F illustrate penetrating members being deployed according to an embodiment of the present invention.

FIG. 2G illustrates removal of the tool of FIG. 2A while leaving the cannula of FIG. 2A in its current location, according to an embodiment of the present invention.

FIGS. 2H-2I illustrate a first mount 104 temporarily connectable to a lower base portion of an implantable port system to mount the lower base portion thereto, according to an embodiment of the present invention.

FIG. 2J illustrates a top view of a port base assembly according to an embodiment of the present invention.

FIG. 2K illustrates a bottom view of the port base assembly of FIG. 2J.

FIG. 2L is a perspective, partial illustration of the deployment tool of FIG. 2A being used to draw/drive penetrating members through tissues, according to an embodiment of the present invention.

FIG. 2M is an isolated, perspective illustration of a penetrating member according to an embodiment of the present invention.

FIG. 2N illustrates mating engagement threads of a port, which are configured and dimensioned to be screwed into threads of an upper base portion, according to an embodiment of the present invention.

FIG. 2O is a schematic, sectional illustration showing a port system having been installed according to an embodiment of the present invention.

FIG. 2P is a more detailed illustration of the system of FIG. 2O.

FIG. 3A is a perspective illustration of a deployment tool according to an embodiment of the present invention.

FIGS. 3B-3C illustrate that, the pre-deployed configuration, the tethers of the system do not have tension applied therethrough, but are slack, according to an embodiment of the present invention.

FIG. 3D shows a series of events that are carried out in deploying a port base using the port base deployment tool of FIG. 3A, according to an embodiment of the present invention.

FIG. 4A is a schematic, partial, perspective illustration of a deployment tool according to an embodiment of the present invention.

FIG. 4B is an enlarged partial view of FIG. 4A better showing the details of tethers, mating receptacles, and recesses.

FIG. 4C is an enlarged, partial, sectional view of FIG. 4B still better showing the details of tethers and mating receptacles.

FIG. 4D illustrates T-bars engaged against a lower base, proximal ends received and mated in mating receptacles, and tethers passing through the suture locks of an upper base, according to an embodiment of the present invention.

FIG. 5A is a perspective view of an alternative embodiment of a tube used in a deployment tool that includes substantially straight needles extending distally from a distal end portion of the tube, according to an embodiment of the present invention.

FIG. 5B is a partial view of the tool of FIG. 5A with a stitcher extended, according to an embodiment of the present invention.

FIG. 6 is a sectional illustration of a port system according to another embodiment of the present invention.

FIGS. 7A-7C schematically illustrate installation of a port system according to various embodiments of the present invention.

FIG. 7D illustrates a disassembled view of the embodiment of FIG. 7A, with the port base having a layer of material that encourages tissue ingrowth on the face thereof facing the base, according to an embodiment of the present invention.

FIG. 8A illustrates a perspective view of an upper base that can be used to install a port system according to another embodiment of the present invention.

FIG. 8B illustrates a port system being installed, according to an embodiment of the present invention.

FIG. 8C illustrates a channel extending radially between openings to receive a tether therein, according to an embodiment of the present invention.

FIG. 8D shows the port system of FIG. 8B having been installed.

FIG. 8E illustrates a distal end portion of a tether driver according to an embodiment of the present invention.

FIGS. 9A-9F illustrate a method of installing a port system according to another embodiment of the present invention.

FIGS. 10A-10C illustrate a method of rapidly installing a port system in fluid communication with a device implanted in the abdominal cavity of a patient according to an embodiment of the present invention.

FIG. 10D illustrates an optional ribbon provided to bridge the opening through the fascia to provide additional connection strength of the assembly, according to an embodiment of the present invention.

FIG. 10E illustrates an embodiment in which a port is built into the base.

FIGS. 10E-10G illustrate an alternative embodiment in which an attachment tab is provided with a plurality of receptacles, and a base has barbed connectors.

FIGS. 11A-11F illustrate a method of rapidly installing a port system in fluid communication with a device implanted in the abdominal cavity of a patient, according to an embodiment of the present invention.

FIGS. 12A-12C illustrate a method of rapidly installing a port system in fluid communication with a device implanted in the abdominal cavity of a patient according to an embodiment of the present invention.

FIG. 12D illustrates the tubular connection between upper base and lower base according to the embodiment of FIGS. 12A-12C.

FIGS. 12E-12G illustrate an embodiment in which a ribbon includes a resorbable component/portion and a non-resorbable component/portion according to an embodiment of the present invention.

FIG. 12H illustrates an alternative arrangement of the port system shown in FIG. 12D.

FIGS. 13A-13F illustrate a method of rapidly installing a port system in fluid communication with a device implanted in the abdominal cavity of a patient according to an embodiment of the present invention.

FIG. 14A shows an instrument configured and adapted to access an incision, tunnel subdermally from the incision to an adjacent location and attach a port according to an embodiment of the present invention.

FIG. 14B illustrates a device and port system having been implanted in a patient according to an embodiment of the present invention.

FIG. 15A illustrates an optional feature that may be used in any of the port devices described herein, and which enables the port (and particularly the septum into which the fill needle is to be inserted) to be visualized by ultrasound imaging, according to an embodiment of the present invention.

FIG. 15B is a longitudinal sectional illustration of a port including the optional feature of FIG. 15A.

### DETAILED DESCRIPTION OF THE INVENTION

Before the present systems, devices and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “an anchor” includes a plurality of such anchors and reference to “the tube” includes reference to one or more tubes and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

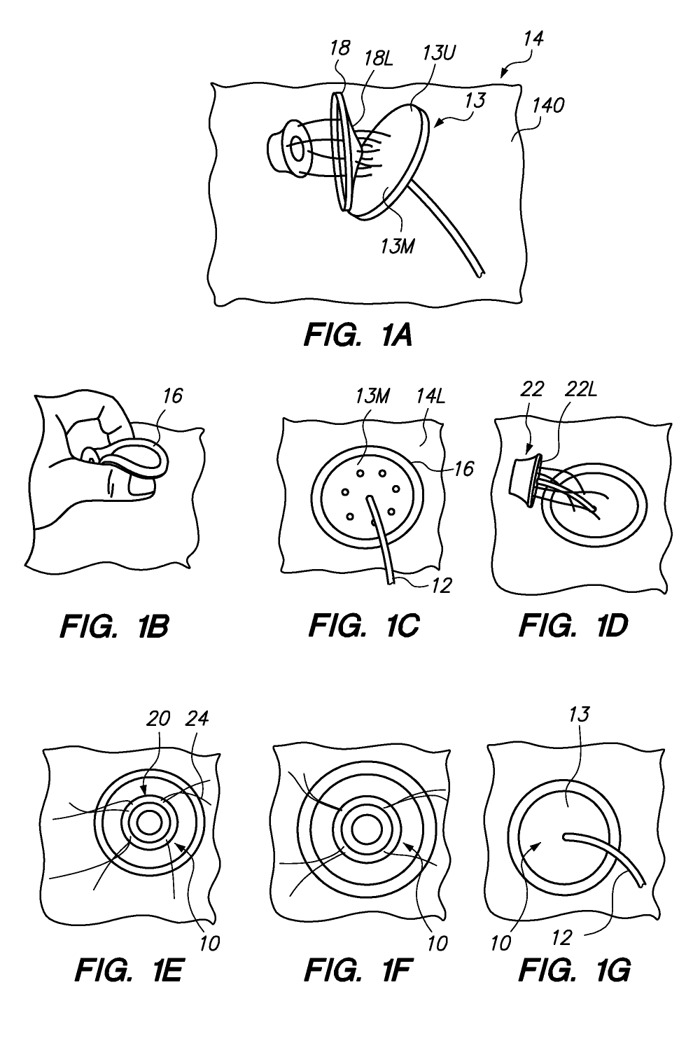

Referring now to the drawings in detail, FIGS. 1A-1G illustrate various stages in a method of attaching an implantable port system 10 to a layer of material 14. Note that the material 14 shown in FIGS. 1A-1G is for illustrative purposes only, and represents one or more tissue layers that would be attached to in a surgical setting. Thus, for example, the system 10 may be attached to the fascia or to an abdominal muscle tissue layer or the like.

The implantable port system 10 includes a base 13 having an upper surface 13U configured to be positioned under and in contact with a first tissue surface in the body, which is represented by the lower surface 14L of the material 14 (see FIG. 1C). The base 13 includes a main body comprising ingrowth material 14M such as DACRON mesh or other polyester multifilament knitted mesh or other material configured and dimensioned to encourage tissue ingrowth into openings in the material. The perimeter of the main body is defined by a flexible ring 16 that allows the base 13 to be reduced in diameter or span dimension, as illustrated in FIG. 1B, and, when released, resiliently returns to its original dimension as illustrated in FIG. 1C. Advantageously, this allows the base 13 to be compressed to the reduced configuration, as shown in FIG. 1B, to pass the base 13 though an opening having a diameter (or other span dimension) that is smaller than the uncompressed diameter of base 13. After being passed through the opening, the compressive forces on the base 13 are released and it resiliently returns to its original, uncompressed dimension, as shown in FIG. 1C. Thus ring 16 helps to maintain the openings in the ingrowth material 13M open and returns the base 13 to its undeformed, uncompressed configuration to overlap the entire perimeter of the opening through which it was passed. Optionally, the surgeon can pull the opening smaller and suture it to have a smaller diameter, in which case the base 13 need only partially cover the opening if desired. Ring 16 may be made of silicone, for example, or other resilient, biocompatible polymer with sufficient resiliency to perform as described. Further alternatively, ring 16 may be formed of resilient metal (spring steel, NITINOL® (nickel-titanium alloy), or the like), optionally coated with silicone or other resilient, biocompatible elastomer.

A second base 18 is provided to cover the opening through the tissue on the upper side, or side opposite where the base 13 is placed. Second base 18 has a lower surface 18L configured to be positioned over the opening and in contact with a tissue surface surrounding the opening on the upper side of the opening. Like base 13, base 18 also has a diameter or other span dimension that is greater than the diameter (or other span dimension) of the opening, so that the base 18 covers the opening upon assembly and implantation of the system.

The lower surface 18L of base 18 comprises ingrowth material which may be the same as the ingrowth material of the upper surface 13U of base 13. Thus the opening is closed off by ingrowth material on both sides when bases 13 and 18 are positioned as described to cover the opening.

An injection port 20 includes a housing 22 having a lower surface that is substantially flat to abut against the upper surface of base 18 when system 10 is assembled for implantation, as shown in FIGS. 1D-1F. In this embodiment, sutures 24 are used to attach the injection port, second base 18 and base 13 together in a sandwich like fashion, which also includes sandwiching the material 14 (or tissue when implanted in a body) between the second base 18 and base 13. Slip knots can be used to draw (FIG. 1E) the components together and maintain them in a sandwiched configuration (FIG. 1F), after which, more secure, permanent knots can optionally be made in the sutures 24 against the base 22 to maintain the desired sandwich configuration. Excess suture material extending proximally from the knots can then be severed from the sutures 24 and discarded. Prior to inserting base 13, the fill tubing 12 connected to the implant to be filled using the port system is threaded through an opening in base 13, through an opening in second base 18 and is connected in fluid communication with port 22. The tubing 12 may be cut to shorten its length, if needed, prior to connecting it to the port 22. FIG. 1G illustrates that the underside of the ingrowth mesh of base 13 is coated with a substantially non-porous material, such as silicone, to prevent adhesions to the underside of the ingrowth layer 13M.

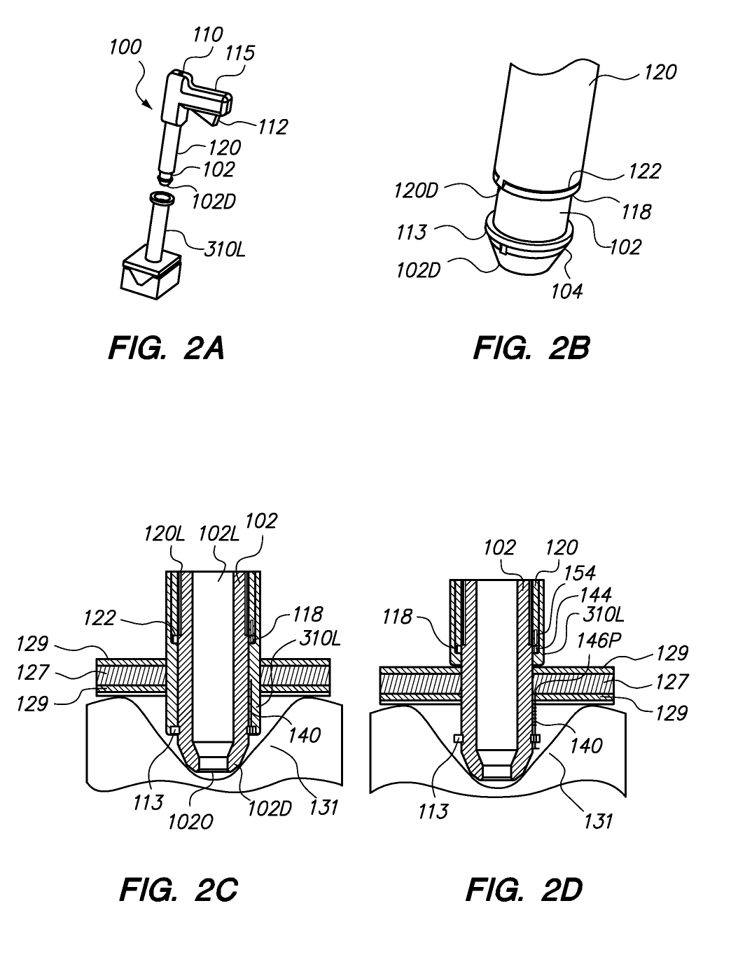

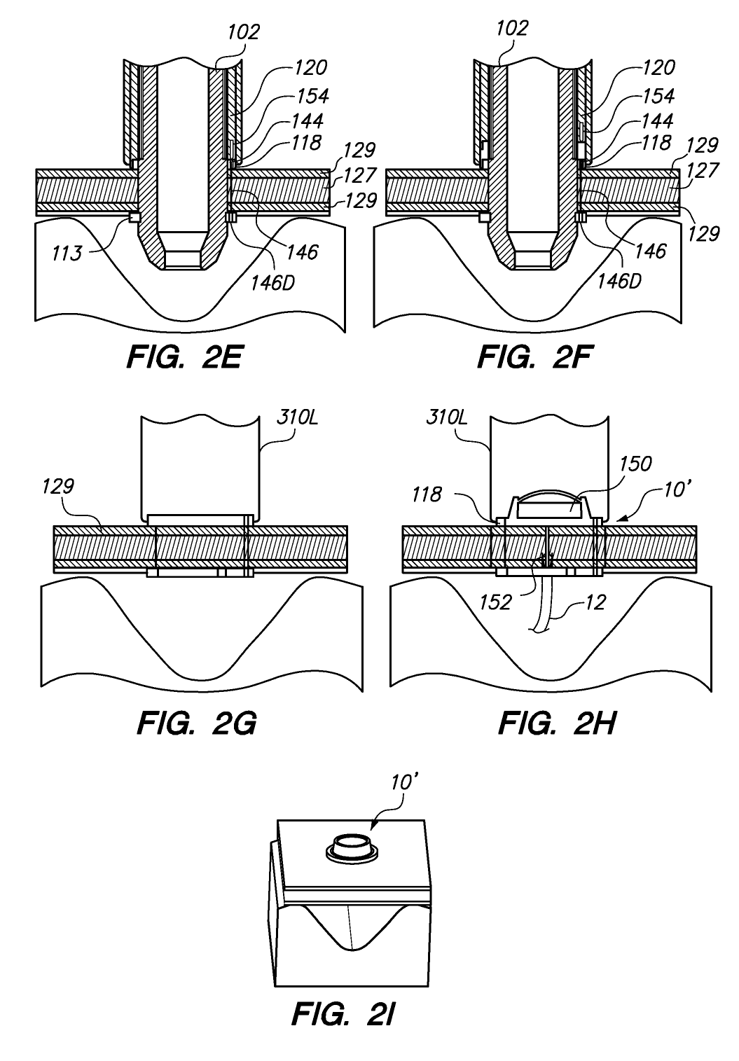

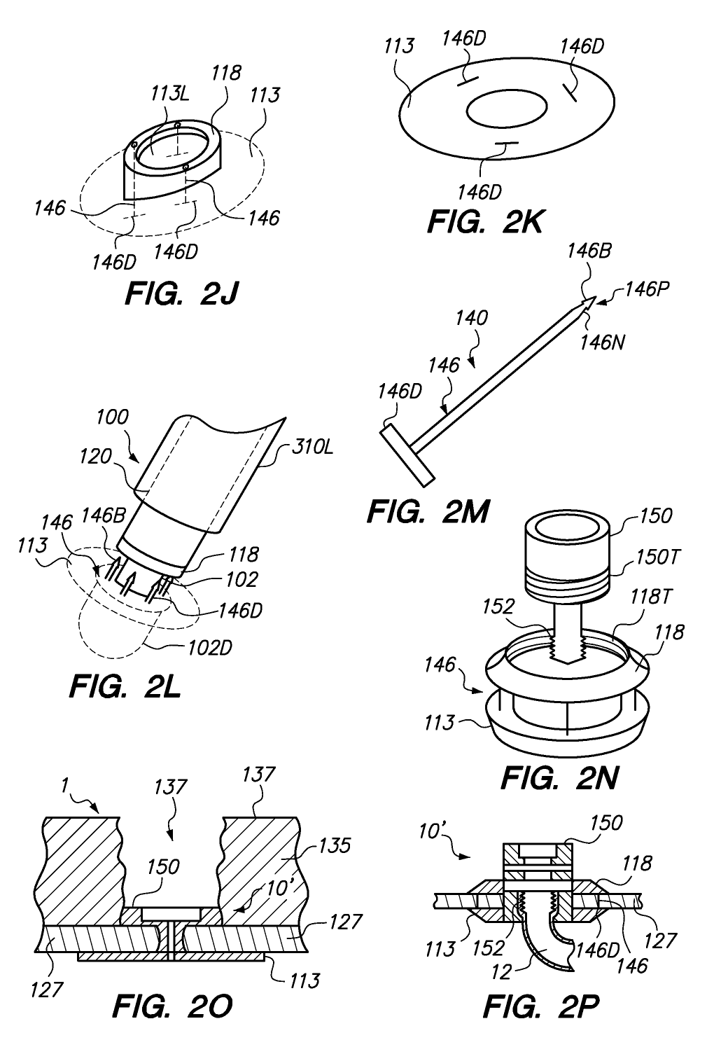

FIG. 2A illustrates a port-base deployment tool 100 according to an embodiment of the present invention. An inner tube 102 extends from a blunt distal end 102D through the proximal portion thereof that extends into tool housing 110 that houses a mechanism (not shown, but which would be readily buildable by one of ordinary skill in the mechanical arts) that includes actuator 112 configured to operate the tool in a manner described below. Tube 102 has a lumen 102L (FIG. 2C) dimensioned to receive an endoscope therein. The blunt distal tip 102D may including an opening 102O therethrough for viewing therethrough by the endoscope and or passing a distal end portion of the endoscope therethrough, or a transparent window may be provided to close opening 102O. Alternatively, or additionally, at least a portion of the blunt distal end 102D may be transparent to allow viewing therethrough, using the endoscope. A first mount 104 (FIG. 2B) is temporarily connectable to a lower base portion 113 of an implantable port system 10′ (FIGS. 2H-2I) to mount the lower base portion 113 thereto, at a location proximal of the blunt distal end 102D.

An outer tube 120 has an open distal end 120D (FIG. 2B) and a lumen 120L (FIG. 2C) dimensioned to slidably receive the inner tube 102 therein. Note that both tubes 102 and 120 have sufficient length to insert their distal ends 102D, 120D into the abdominal cavity while the proximal end portions extending into housing 110 are located out of the patient, proximal of the patient's skin. The outer tube 120 includes a second mount 122 configured to temporarily connect to an upper base portion 118 of the port system 10′ to mount the upper base portion 118 thereto, such that, when the port implantation system 10′ is assembled, the upper base portion 118 is proximal of the lower base portion 113.

FIG. 2A schematically illustrates a procedure where an expandable implant has been placed and attached in the abdominal cavity of a patient and commencement of placement and attachment of the port system 10′. At FIG. 2A, the port-base deployment tool is inserted into the cannula 310L that extends into the abdominal cavity. Prior to insertion, the base deployment tool is advanced over the fill tubing 12 (not shown) that is in fluid communication with the implanted expandable device and that extends out of the abdominal opening and out of the patient. The tubes 102, 120 are advanced into the cannula, preferably while viewing the advancement via an endoscope (e.g., 5 mm endoscope) inserted into lumen 102L. As the blunt tip 102D exits the distal end of the cannula 310L it compresses the bowel 131 as illustrated in FIG. 2C, thereby moving it out of the way to minimize risk of puncturing, pinching or otherwise damaging or obstructing the bowel during the port attachment procedure. The tool is inserted until the “notches” formed between bases 113 and 118 extend across the fascia and abdominal muscle layers 127, 129 and base 113 is in the abdominal cavity while base 118 is external of the abdominal cavity as illustrated in FIG. 2C.

Next, the cannula 310L is retracted so that the distal end thereof exits the abdominal cavity, clearing the abdominal wall (abdominal muscle 129 and fascia 127). This allows the abdominal wall surrounding the now exposed tube 102 to elastically return towards its undeformed conformation, thereby impinging upon the outer surface of the tube 102, as illustrated in FIG. 2D, and thereby moving the tissue 127, 129 into the paths of the penetrating members 140, which may be barbed needles or the like. Note that although only one penetrating member 140 is shown in FIGS. 2D-2F, this is because sectional views are illustrated. Typically, a plurality of such penetrating members 140 are provided, such as three, four or more (although two may also be employed), spaced evenly, circumferentially about the bases 113. Likewise, mating receptacles 142, 144 are provided in base 118 and tube 120 in locations corresponding to the locations of penetrating members 140 in base 113, the functionality of which are described below.

Next, the operator actuates the actuator 112, such as by squeezing it toward the handle 115 in the embodiment of FIG. 2A. This drives the bases 113 and 118 towards one another by drawing distal tip 102D proximally relative to tube 102. Alternatively, tube 120 can be driven distally relative to distal tip 102D or distal tip 102D can be driven proximally and tube 120 can be driven distally. This also causes the penetrating members 140 to be driven through the tissues 127, 129, through the receptacles 142 in base 118 and into engagement with mating, locking receptacles 144 in tube 120, as illustrated in FIGS. 2E-2F. As the actuator reaches near midpoint of its travel, the distal ends 146D of tethers 146 are drawn against base 113 and function as anchors and the tethers 146 are held under tension while a cutting mechanism (not shown) is actuated to sever the tethers from the penetrating members 140. As the actuator 112 nears the end of its travel, the bases 113, 118 are disengaged from the mounts 104, 122 and from tubes 102, 120 and the tool 100 is removed while leaving the cannula 310L in its current location abutted up against (or in near proximity) to the external abdominal wall surface 129, as illustrated in FIG. 2G. Tubing 12 is connected to the distal connector 152 of port 150, and port 150 is then advanced down the cannula 310L and snapped into the base 118 (or attached by alternative attachment techniques, such as screwing, or the like) as illustrated in FIG. 2H. FIG. 2I illustrates the port system 10′ at the completion of the attachment procedure, after removal of cannula 310L. Following this, the surgeon can then completely close the patient by closing the opening through the skin and thereby concealing the port. FIG. 2J illustrates a top view of the port base assembly (i.e., external of the patient, viewing base 118) after installation thereof, but prior to installation of the port 150. This is how the assembly would appear if cannula 310L were removed in FIG. 2G, with a top perspective view taken, rather than a sectional view. The main lumen 113L is configured and dimensioned to receive and connect to the port 150. Likewise, FIG. 2K illustrates a bottom view of the port base assembly (i.e., view from inside the abdominal cavity, looking up, viewing the bottom of base 113) after installation thereof. The distal end anchors 146D (in this case, T-bars, but could alternatively be hooks, balls, washers or some other type of enlargement) can be seen abutted against the lower surface of base 113. The main lumen 113L allows passage of the fill tube 12 (not shown in FIG. 2K and any portion of the port 150 that might extend below the level of the base 113.

FIG. 2L is a perspective, partial illustration of the deployment tool 100 being used to draw/drive the penetrating members 140 through the tissues and towards the receptacles in base 118. FIG. 2M is an isolated, perspective illustration of penetrating member 140 comprising tether 146 having a proximal end portion 146P including a barb 146B and a neck portion or otherwise reduced diameter portion 146N that is engaged by mating receptacles 144 in tube 120. The distal end portion includes an anchor 146D formed at a distal end of tether 146 which, in the embodiment of FIG. 2M is a T-bar, but could alternatively be a hook, ball, washer or some other type of enlargement).

FIG. 2N illustrates the mating engagement threads 150T of port 150, which are configured and dimensioned to be screwed into threads 118T of upper base portion 118. FIG. 2O is a schematic, sectional illustration showing the port system 10′ having been installed as described above with regard to FIGS. 2A-2I, and before closure of the access opening through the skin 137 and fatty tissue 135. FIG. 2P is a more detailed illustration of the system 10′ of FIG. 2O, which shows tubing 12 connected to the distal connector 152 of port 150 and extending distally into the abdominal cavity where it is in fluid communication with an implant (not shown).

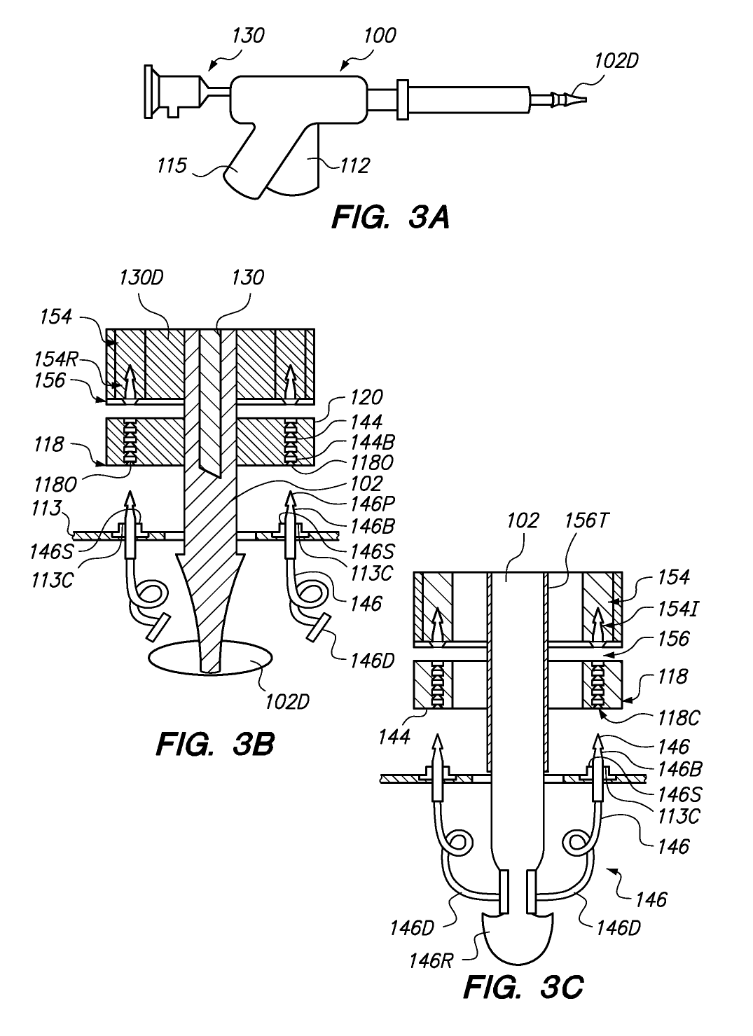

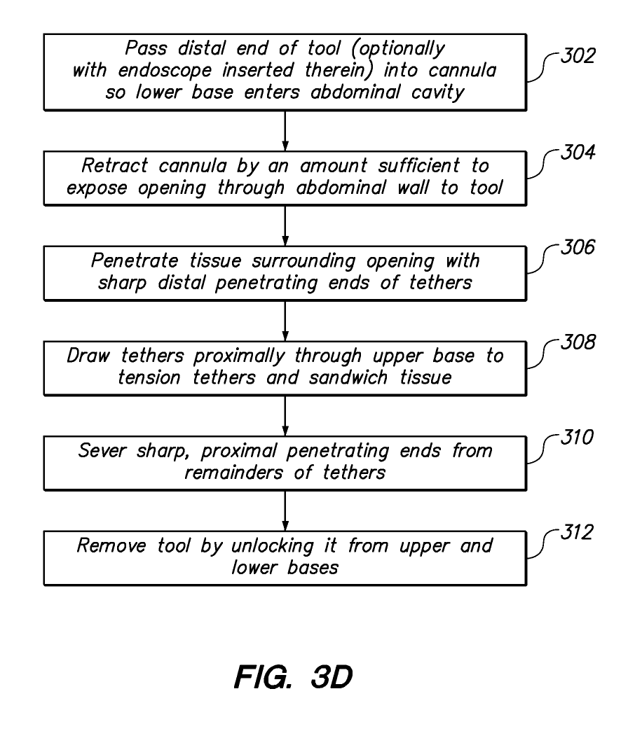

FIG. 3A is a perspective illustration of a deployment tool 100 showing an endoscope 130 having been inserted within tube 102. Tube 102 is transparent and/or includes windows in at least a distal end portion thereof to allow viewing therethrough by a distal end portion 130D of endoscope 130, as illustrated in FIG. 3B. Additionally, the endoscope 130 can be used to view out of the distal end 102D of tool 100. The distal ends 146D (in this embodiment, T-bars) are stowed against the distal end portion 102D of tool 100 and may be temporarily held within recesses 146R as shown in FIG. 3C, prior to drawing tension of the tethers 146. Thus, in the pre-deployed configuration shown in FIGS. 3B and 3C, the tethers 146 do not have tension applied therethrough, but are slack.

FIG. 3D shows a series of events that are carried out in deploying a port base using port base deployment tool according to an embodiment of the present invention. At event 302, the distal end of the tool 100 (optionally with an endoscope 130 inserted therein) is inserted into a cannula 310L (e.g., see FIG. 2A) until the lower base 113 mounted on tool 100 enters the abdominal cavity (e.g., see FIGS. 2C, 3B, 3D). At event 302, the cannula 310L is retracted by an amount sufficient to expose the tissues surrounding the opening leading into the abdominal wall to the tool 100 (tube 102), e.g., see FIG. 3D. The resilient nature of the abdominal muscle and other surrounding tissues causes them to resiliently return towards closing the opening, thus the tissues abut up against the tool 100/tube 102. Next, the actuator 112 is actuated to draw tube 102 proximally, thereby driving the proximal, penetrating ends 146P through the tissues abutted around tube 102 as illustrated in FIG. 2D. Further actuation of the tool 100 draws tube 102 further proximally and enlarged portions/barbs 146B of the proximal ends 146P of tethers 146 engage in receptacles 154 with necks 154R of the receptacles 154 preventing the enlarged portions/barbs 154B from passing back out distally or the receptacles 154. Thus, tube 120 is then drawn proximally relative to tube 102 to draw the proximal ends 146P of tethers 146 proximally and thereby apply tension to tethers 146, event 308. This draws the lower base 113 against the upper base 118, thereby sandwiching the tissues therebetween.

Next, at event 310, the sharp proximal penetrating ends 116B are severed by actuation of the cutter 156. In the embodiment of FIGS. 3B-3C, cutter 156 rotates to sever the tethers 146. A cutter tube 156T (see FIG. 3C) is rotatable by the operator (either by the end drive action of the actuator 112, or by a separate rotary control) to rotate the cutter 156 to sever the tethers 146. Once severed, the sharp proximal penetrating ends 146B remain in the mating receptacles of tube 120, as neck regions 154R mate with the necks 146N of the sharp proximal penetrating ends 146B. At the same time, the portions of the tensioned tethers that extend through the openings 1180 in the mating receptacles/suture locks 144 in upper base 118 are pierced by retention members 144B (see FIGS. 3B-3C). This penetration prevents the tethers from backsliding distally away from receptacle 144 and therefore maintains the tension in the tethers.

At event 312, tool 100 is removed from the patient by disengaging it from the upper and lower bases 118, 113 by simply pulling on the tool 100 and sliding the tool 100 axially out of the patient.

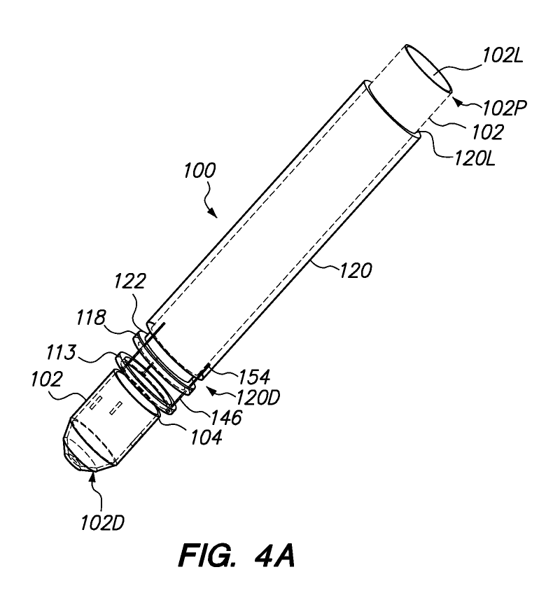

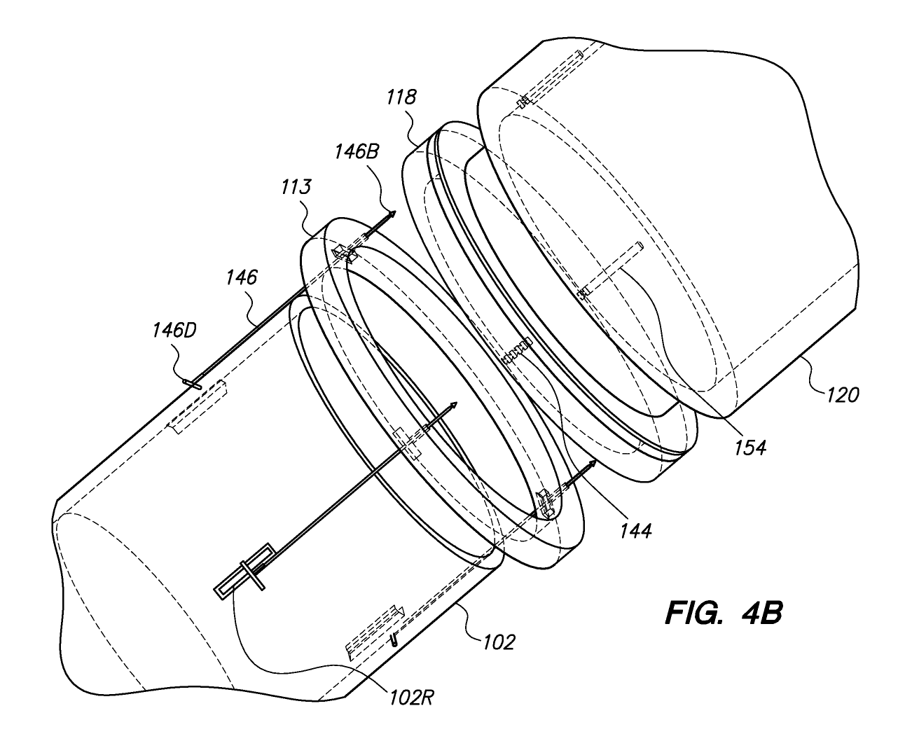

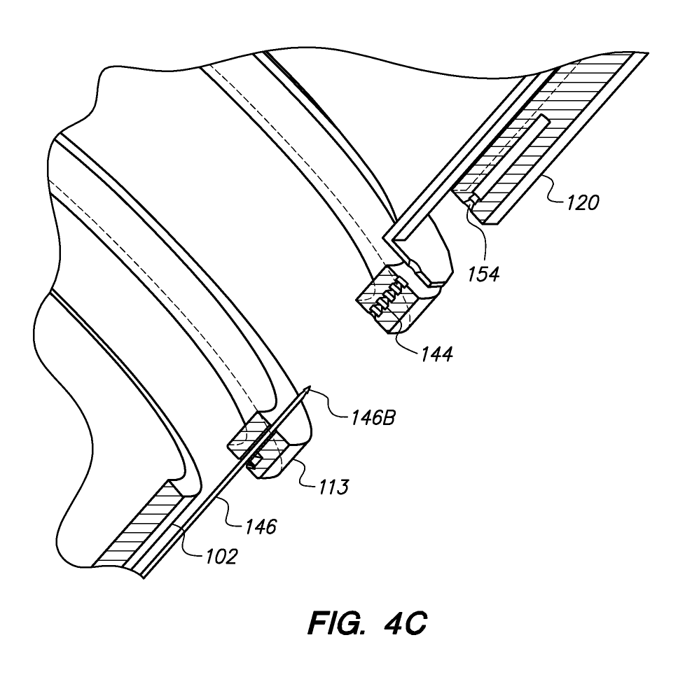

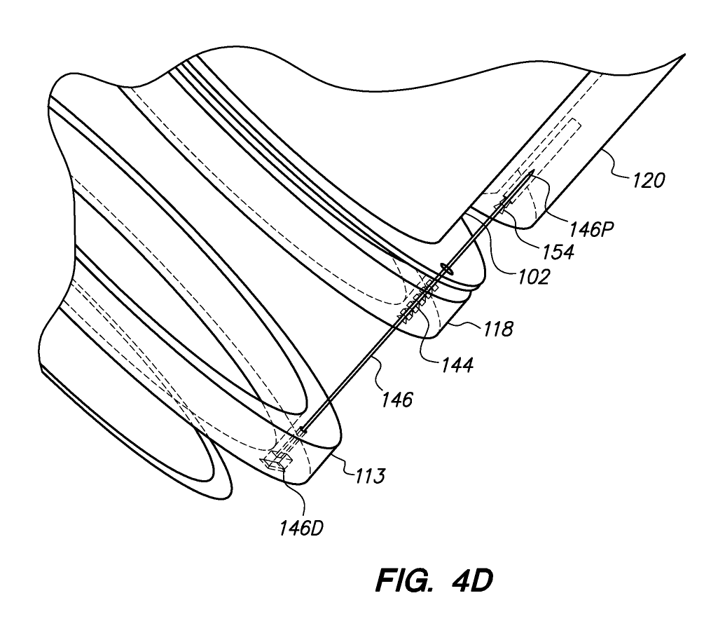

FIG. 4A is a schematic, partial, perspective illustration of tool 100 that shows the relationship between tubes 102 and 120. The portion of tube 102 that extends between bases 113 and 118 has been cut away to better show the relationship between the bases 113, 118, tethers 146, receptacles 154 and tubes 102, 120. FIG. 4B is an enlarged partial view of FIG. 4A better showing the details of tethers 146, mating receptacles 144, 154 and recesses 102R that temporarily stow the distal ends 146D of tethers 146. FIG. 4C is an enlarged, partial, sectional view of FIG. 4B still better showing the details of tethers 146 and mating receptacles 144, 154. FIG. 4D illustrates T-bars 146D engaged against lower base 113, proximal ends 146P received and mated in mating receptacles 154, and tethers 146 passing through the suture locks 144 of upper base 118.

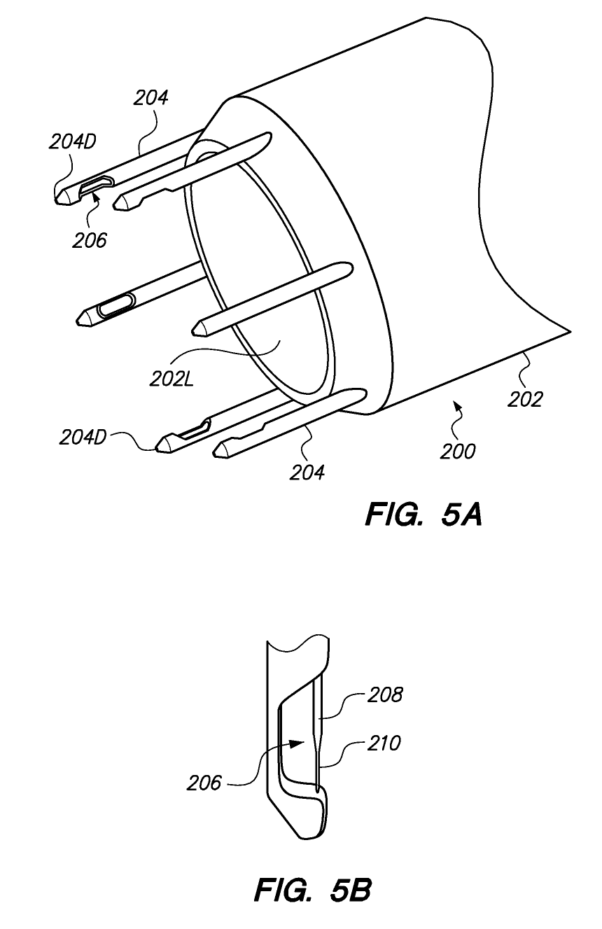

FIG. 5A is a perspective view of an alternative embodiment of a tube 202 used in a deployment tool 200 that includes substantially straight needles 204 extending distally from a distal end portion of tube 202. Each of the needles 204, includes a recess 206 at a distal end portion thereof Upon insertion of needles 204 through tissue surrounding an opening to be closed and/or to attach a port to, the recesses 206 are placed in alignment with the tissue surrounding the opening which has been pierced by the needles 204. The tissue that has been pierced resiliently rebounds into recesses 206. Tool 200 is then operated to distend stitchers 208 that pass back through (in a proximal direction) the tissue within the recesses, thereby drawing sutures 210 therethrough and placing a stitch/suture in the tissues at each of the locations 206. These sutures 210 can then be used to attach one or more port bases 113, 118 thereto, or can be used to draw the portions of tissue together so as to close the opening in the patient.

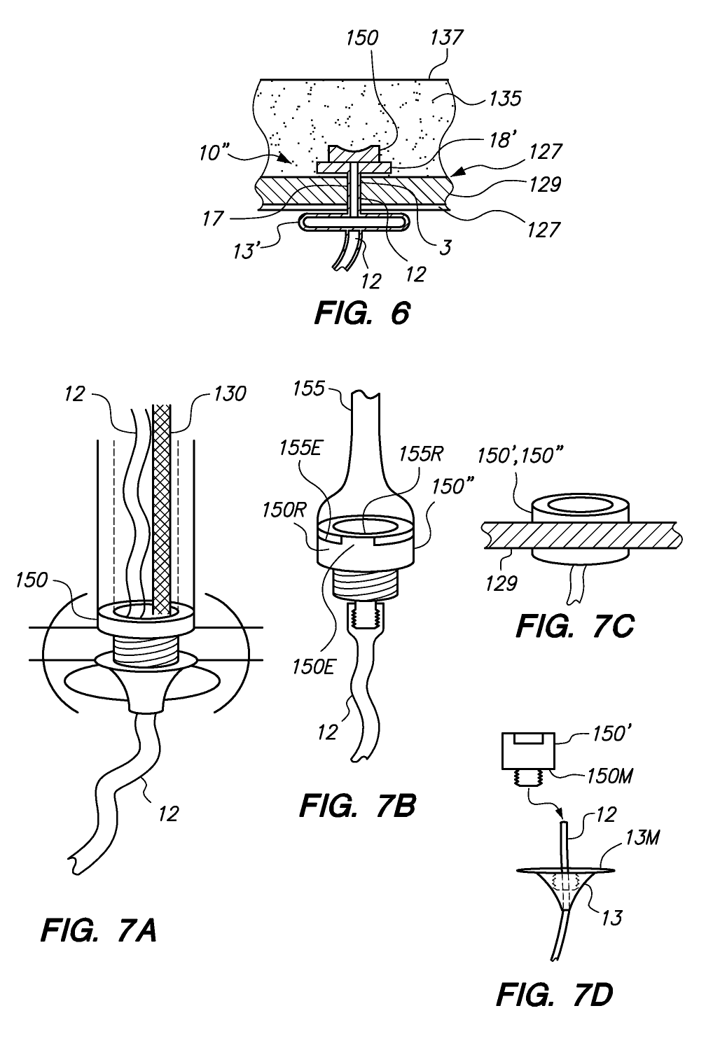

FIG. 6 is a sectional illustration of a port system 10″ according to another embodiment of the present invention. In this embodiment, lower base 13′ is an inflatable base, such as a balloon, bladder or other inflatable member that can be deflated to pass through the opening 3 through the fascia 129 and abdominal muscle 129. Once positioned in the abdominal cavity, base 13′ can then be inflated to a size that is too large to pass back through the opening 3, as illustrated in FIG. 6. Upper base 18′ may be connected to lower base 13′ via tubing 12, and/or inflation tubing 17 used to inflate the balloon 13′. Additionally or alternatively, a porous surface or layer to encourage tissue ingrowth 18L, 13U may be provided on one or both of the lower surface of base 18′ and the upper surface of base 13′. Further additionally or alternatively, tube 17 may be coated with a tissue ingrowth encouraging layer or a porous ribbon may be placed to extend between and connect bases 13′, 18′. FIG. 6 illustrates the port system 10″ after completed installation thereof, including closing the opening through the fatty tissue 135 and skin 137. The balloon 13′ may be expandable by inflation using saline, gas or other biocompatible fluid, or mechanically, such as by a self-expanding silicone structure, or other self-expanding mechanical structure for example.

FIGS. 7A-7C schematically illustrate installation of a port system according to various embodiments of the present invention. FIG. 7A illustrates a port base 150 being threaded into a base 13. As shown in FIG. 7D, the port base 150′ may have a layer of material 150M that encourages tissue ingrowth on the face thereof facing the base 13. Port base 150′ screws onto base 13 with the port base 150′ and base 13 being located on opposite sides of the muscle/fascia to which they are being attached. Optionally, an endoscope 130 can be used to see into and through the opening in which the port is being installed. Fill tubing 12 extends up through the hollow bases 13, 150′ as shown in FIG. 7A.

FIG. 7B illustrates a driver tool 155 that is configured to engage and drive port 150″. Recesses 155R and extensions 155E of driver tool 155 engage and mate with extensions 150E and recesses 150R of port 150′ so that tool 155 can be used to apply torque to port 150′ without slippage and thereby effectively drive the screwing of the port 150′ into secure placement during installation. Alternatively, driver tool 155 may be used to push port 150″ into secure placement via press fitting. FIG. 7C illustrates port 150′, 150″ having been installed so that its proximal face is accessible proximally of the abdominal muscle 129.

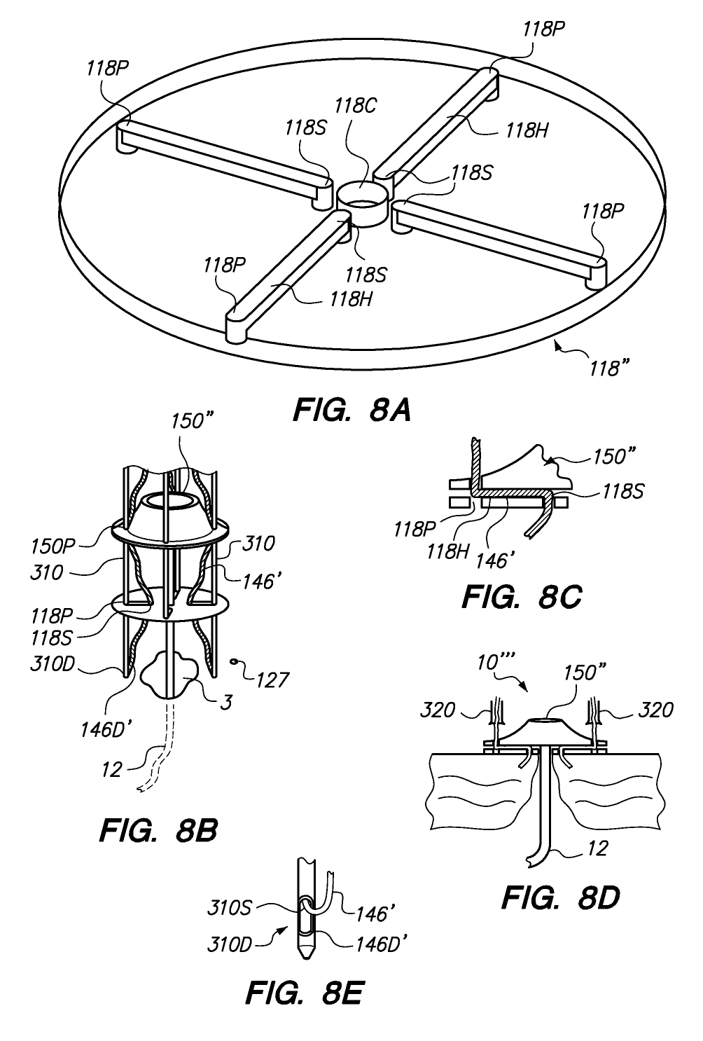

FIG. 8A illustrates a perspective view of an upper base 118″ that can be used to install a port system according to another embodiment of the present invention. Alternatively, the opening 118C through base 118″ may be closed in instances where upper base 118″ is used for wound closure, such as to close a hernia or the like. It is further noted that the other base systems described herein can likewise be modified to close the opening otherwise used to insert port 150, so as to be used for a wound closure system. Base 118″ incudes through hole 118C to allow port 150″ to be connected to tubing 12, as illustrated in FIG. 8D. Base 118″ further includes a set of peripheral through holes 118P and a set of secondary through holes 118S radially aligned with peripheral through holes 118P, respectively and located radially inward of through holes 118P. FIG. 8B illustrates an arrangement of the components of the port system 10′ arranged for implantation, over an opening 3 through the abdominal wall. This arrangement will typically be delivered through a tube such as cannula 310L, but cannula 310L is not shown here for clarity in viewing the other components.

Tether anchor drivers 310 are inserted through peripheral openings 150P of port 150″ (which align with peripheral openings 118P), and peripheral openings 118P of base 118″ as shown in FIG. 8B. The distal ends of distal end portions 310D of tether anchor drivers 310 are sharpened to facilitate piercing through the tissues, including the fascia and abdominal muscle. The distal end portions are further slotted, or otherwise configured to each releasably hold an enlarged distal end portion 146D′ of a tether 146′. In the embodiment shown in FIG. 8E, enlarged portion 146D′ is a T-bar. However, other forms of enlarged end portions, including, but not limited to those identified above, could be substituted for the T-bars.

Tethers 146 are temporarily fixed to distal end portions 310D of tether anchor drivers 310, respectively, in a manner as already described. Tether 146 is further threaded through secondary opening 118S and opening 150P as shown in FIG. 8B. A channel 118H extends radially between opening 118S and opening 118P to receive tether 146 therein, e.g., see FIG. 8C. The assembly/arrangement shown in FIG. 8B is placed into cannula 310L after the anterior fascia 127 has been exposed by forming an opening through the skin and fatty tissues. The tether anchor drivers 310 are driven though the fascia 127 to a controlled depth (to prevent driving too far and potentially piercing the bowel) and then withdrawn. Upon withdrawal of the tether anchor drivers 310, the enlarged distal end portions (anchors, i.e., T-bars, in this embodiment) 146D′ remain anchored in the tissue, slide out of the slots 310S and therefore anchor the tethers 146 to the tissue.

Each tether 146 is routed through a knot tie ferrule 320 (FIG. 8D) and pulled until cinched to a predetermined tension level in the tether 146 has been achieved. This pulling and cinching action draws the tissues (anchored by anchor 146D′) toward openings 118S, thereby drawing the tissues toward the center of opening 3 causing the tissue to close up around the opening, as can be seen when comparing FIG. 8B to FIG. 8D. With the tissues drawn up around tubing 12 and opening 3 closed or substantially narrowed, the cinching also draw the port 150″ against base 118″ and draws base 118″ against the anterior surface of the fascia 127, thereby closing the opening and installing the port system 10″′ as shown in FIG. 8D. The excess tether extending proximally of the ferrules can be severed and removed. The ferrule act as suture locks, similar to that described above with regard to FIG. 3, reference number 144.

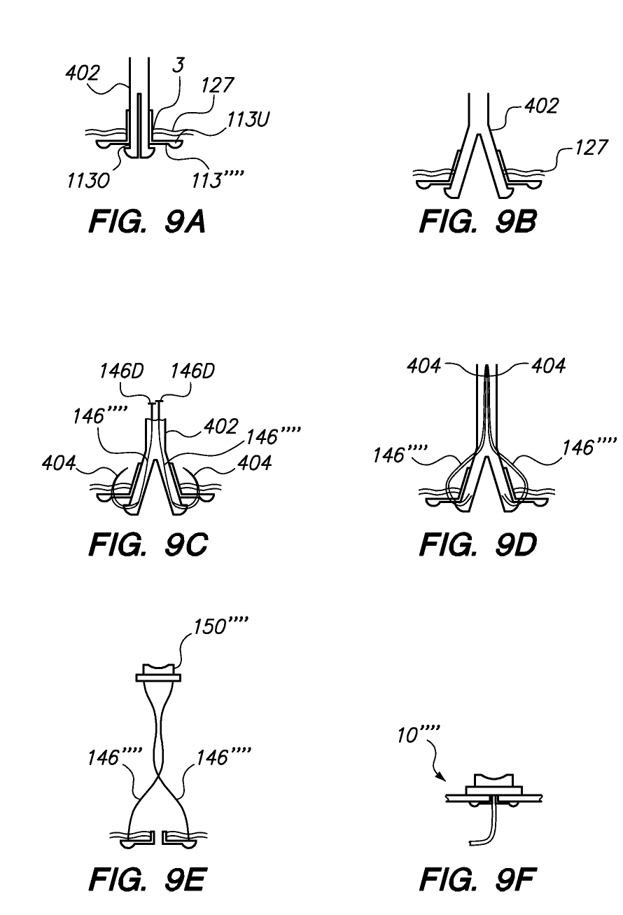

FIGS. 9A-9E illustrate a method of installing a port system 10″″ according to another embodiment of the present invention. In FIG. 9A, a lower base 113″″ is inserted through an opening 3 in a patient. Lower base 113″″ is flexible and resiliently expandable, and may be made of silicon, or the like, for example. A coating or layer 113U configured to encourage tissue ingrowth may be place on the upper surface of lower base 113″″. An expansion tool 402 inserted through the opening 1130 of base 113″″ is expanded as illustrated in FIG. 9B and needles 404 are deployed proximally from a working end of tool 402, as illustrated in FIG. 9C, to drive needles 404 through base 113″″ and through the fascia 127 (and, optionally, the abdominal muscle). The needles 402 track back into tool 402 in FIG. 9D, thereby routing tethers 146″″ back to the operator. Needles 404 are then removed and tool 402 is removed. Port 150″″ is then threaded over over the tethers 146″″ through peripheral holes 150P in the port 150″″ and port 150″″ is secured over the opening 3 as illustrated in FIG. 9F using a securement mechanism such as 144 or 320.

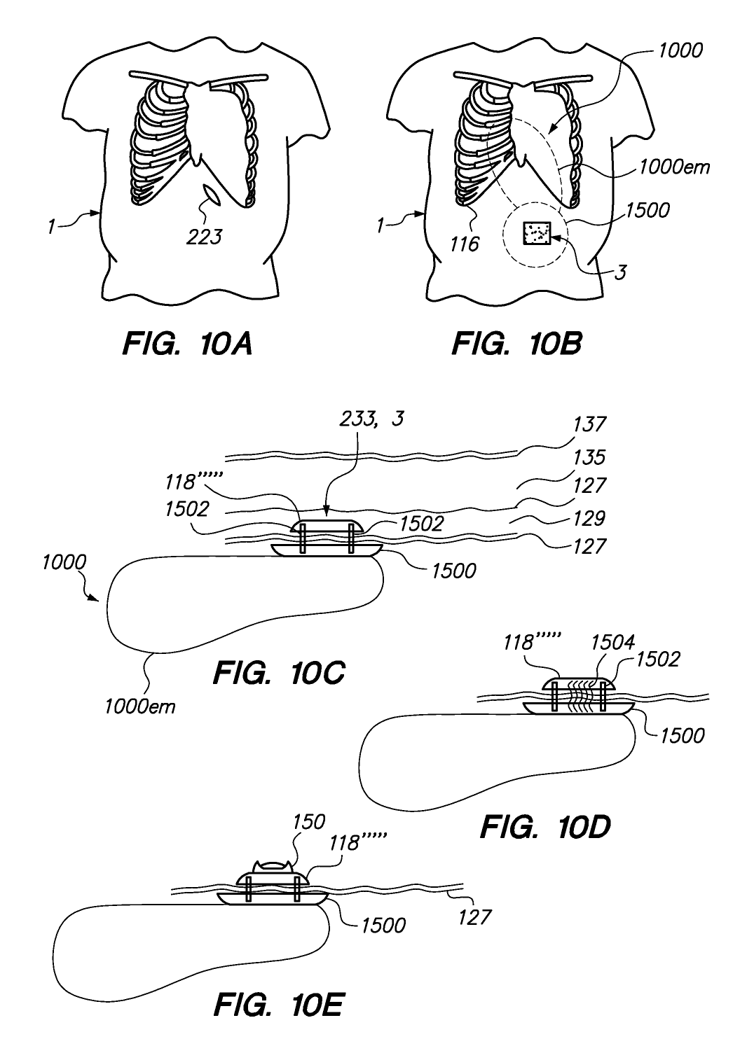

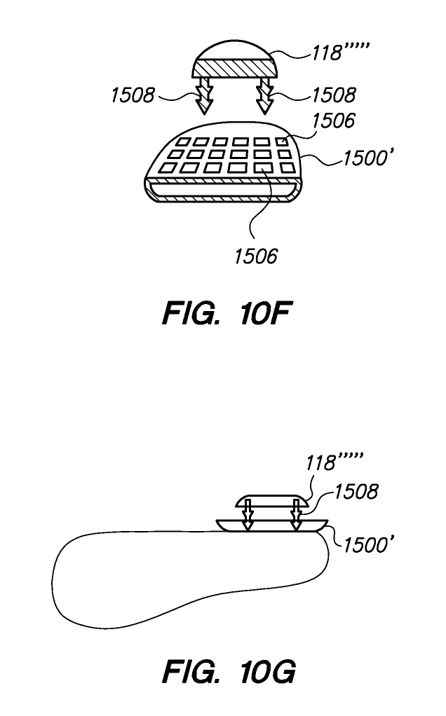

FIG. 10A-10C illustrate a method of rapidly installing a port system in fluid communication with a device implanted in the abdominal cavity of a patient 1 according to an embodiment of the present invention. An incision 223 is made in the abdominal region of the patient 1 through which an opening leading into the abdominal cavity of the patient is to be established to deliver the device 1000. After forming an opening 3 from the incision 223 leading into the abdominal cavity as noted, the device 1000, including expandable member 1000em and attachment tab(s) 1500 are inserted through the opening 3 and into the desired position in the abdominal cavity as illustrated in FIG. 10B. In this embodiment, the attachment tab(s) 1500 include attachment needles 1502 that extend proximally from attachment tab(s) and, once positioned, penetrate the fascia 127 and the sharp tips anchor into an upper base 118″″′ above the fascia 127 (and optionally above the abdominal muscle, 129, as the port can be located below or above the abdominal muscle 129), thereby sandwiching the fascia (or fascia and abdominal muscle). The port 150 can then be attached to base 118″″′ by any of the techniques described above, or can be built into the upper base. Optionally, a ribbon 1504 made of material that encourages tissue ingrowth can be provided to bridge the opening 3 through the fascia to provide additional connection strength of the assembly by increasing the tissue ingrowth therein, as well as further mechanically linking tab 1500 to base 118″″′, see FIG. 10D. FIG. 10E illustrates an embodiment in which a port 150 is built into the base 118″″′. FIGS. 10E-10G illustrate an alternative embodiment in which attachment tab 1500 is provided with a plurality of receptacles 1506, and base 118″″′ has barbed connectors 1508 extending distally from a lower surface thereof. In this embodiment, base 118″″′ is pressed down on the fascia 127 to penetrate the fascia 127 with barbed connectors 1508, after which barbed connectors are received in an mate with receptacles 1506, as illustrated in FIGS. 10E-10G. The barbs of the barbed connectors 1508 are angled so that they are allowed to pass into a receptacle 1506, but are prevented from pulling back out of the receptacle 1506.

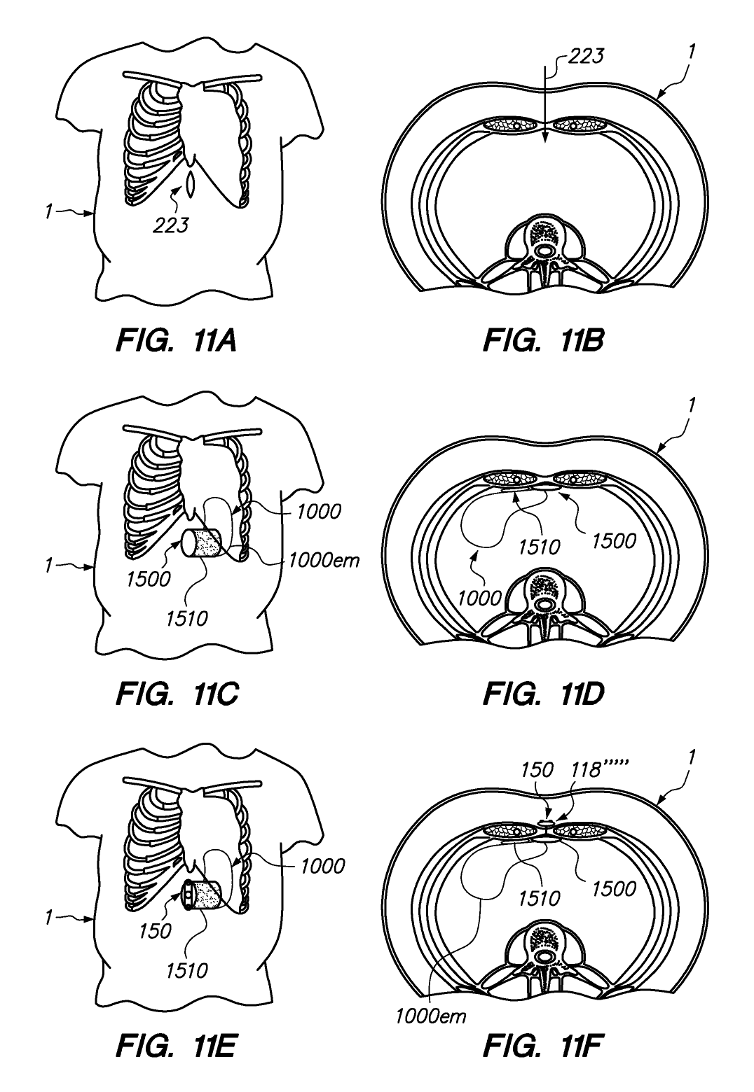

FIGS. 11A-11F illustrate a method of rapidly installing a port system in fluid communication with a device 1000 implanted in the abdominal cavity of a patient 1 according to an embodiment of the present invention. An incision 223 (see the frontal illustration of FIG. 11A and the sectional illustration of FIG. 11B) is made in the abdominal region of the patient 1 through which an opening leading into the abdominal cavity of the patient is to be established to deliver the device 1000. After forming an opening 3 from the incision 223 leading into the abdominal cavity as noted, the device 1000, including expandable member 1000em and attachment tab(s) 1500 are inserted through the opening 3 and into the desired position in the abdominal cavity as illustrated in FIGS. 11C-11D. In this embodiment, the attachment tab(s) 1500 are provided with a coating or layer of material 1510 adjacent the location where the port is to be connected, that encourages tissue ingrowth.

The upper base (such as upper base 118″″′ in FIG. 10F is attached to tab (lower base) 1500 in a manner as described earlier, and a port 150 is either built into base 118″″′ or is subsequently attached in a manner as described previously, see FIGS. 11E-11F.

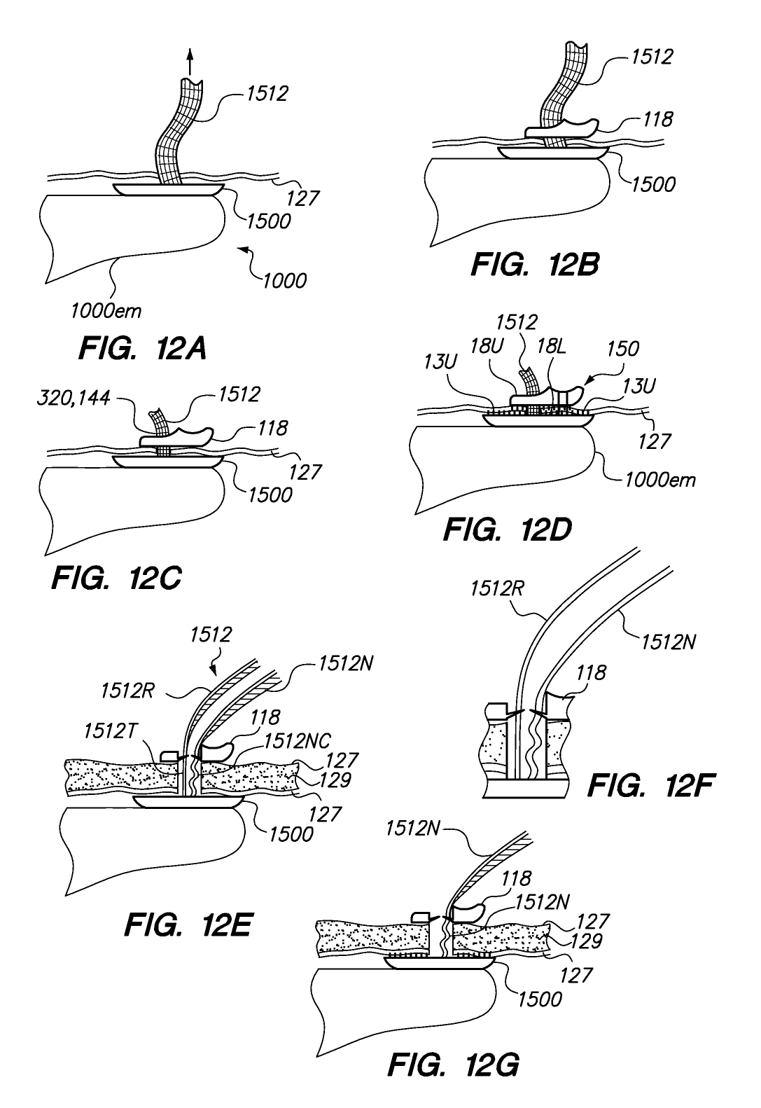

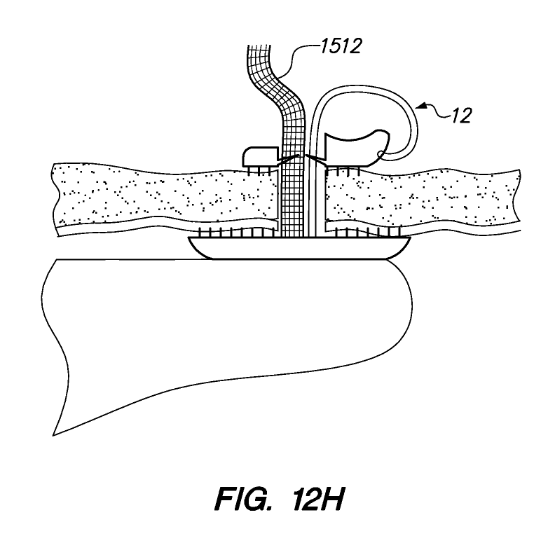

FIGS. 12A-12C illustrate a method of rapidly installing a port system in fluid communication with a device 1000 implanted in the abdominal cavity of a patient according to an embodiment of the present invention. In this embodiment, lower base/attachment tab 1500 includes a ribbon 1512 extending proximally from an upper surface thereof as shown in FIG. 12A. Once the implant has been inserted into the abdominal cavity and expanded, the installer pulls on the ribbon 1512 (which is already extending out of the opening 3 and incision 223) to draw the lower base 1500 up against the fascia. Additional tools may be used (including, but not limited to an endoscope) to provide visual confirmation that the fascial space is clear before the base 1500 is drawn up against it. Additional tools may also be employed to help orient the device 1000 prior to attachment of the port 150 and/or drawing the base 1500 against the fascia 127. Once the lower base 1500 has been drawn against the fascia 127, the upper base 118 (18, 118′, 118″m 118″′, 118″″, 118″″′, use of any of these reference numerals is interchangeable with the others in this description, unless the particular application would prevent it) is slid down over the ribbon 151 and into contact with the fascia or abdominal muscle, thereby sandwiching the tissues between the upper and lower bases, as illustrated in FIG. 12B. In this instance, upper base 118 includes a ferrule 320 or suture lock 144 that maintains the tension on ribbon 1512 and prevents it from backsliding distally through the ferrule/suture lock (speed nuts, or similar feature) 320, 144. The excess ribbon extending proximally from upper base 118 is then trimmed as illustrated in FIG. 12C. The mechanical connection between the upper and lower bases 118, 1500 anchors the device 1000 while tissue ingrows into the ribbon 1512 (as well as the lower surface of upper base 118 and upper surface of lower base 1500 when provided with tissue ingrowth surfaces).

FIG. 12D illustrates the tubular connection 12 between upper base 118 and lower base 1500 that place port 150 (integral with upper base 118 in this embodiment) in fluid communication with expandable member 1000em. FIG. 12D also illustrates the tissue ingrowth enhancing surfaces 18L and 13U providing on the bottom of upper base 18 and top of lower base 1500, respectively.

The material making up ribbon 1512 can be selected to encourage tissue ingrowth, and to allow it to absorb completely or partially over time, to allow resulting length of the ribbon to increase. Examples of absorbable materials that may be employed include, but are not limited to: BIOSYN™ (synthetic polyester) or Gore SEAMGUARD™ (polyglycolic acid and trimethylene carbonate). Examples of non-absorbable materials that may be employed include, but are not limited to: PET (polyethylene terephthalate) mesh or PTFE (polytetrafluoroethylene, e.g., GORE-TEX™ or the like). FIGS. 12E-12G illustrate an embodiment in which ribbon 1512 includes a resorbable component/portion 1512R and a non-resorbable component/portion 1512N. FIG. 12E illustrates that the nonresorbable portion 1512N is initially configured in a convoluted or non-tensioned orientation 1512NC even when the resorbable portion 1512R is placed under tension 1512RT by drawing ribbon 1512 through upper base 118 in a manner as described above, to sandwich the tissues, e.g., see FIG. 12F. After a period of time, the resorbable portion 1512R resorbs, thereby releasing the original amount of tension that held the bases 118, 1500 under tension as in FIGS. 12E-12F. This reduced tension allows expansion of the space between the bases 118 and 1500 until the non resorbable component is brought under tension, at a reduced level relative to the original amount of tension on the resorbable component, see FIG. 12G. Tissue ingrowth to the base (or bases) and non-resorbable portion 1512N create long-term attachment of the port system and implant to the abdominal wall.

FIG. 12H illustrates an alternative arrangement of the port system shown in FIG. 12D. In this alternative arrangement, tubing 12 is routed through the same opening that ribbon 1512 is routed, alongside of ribbon 1512.

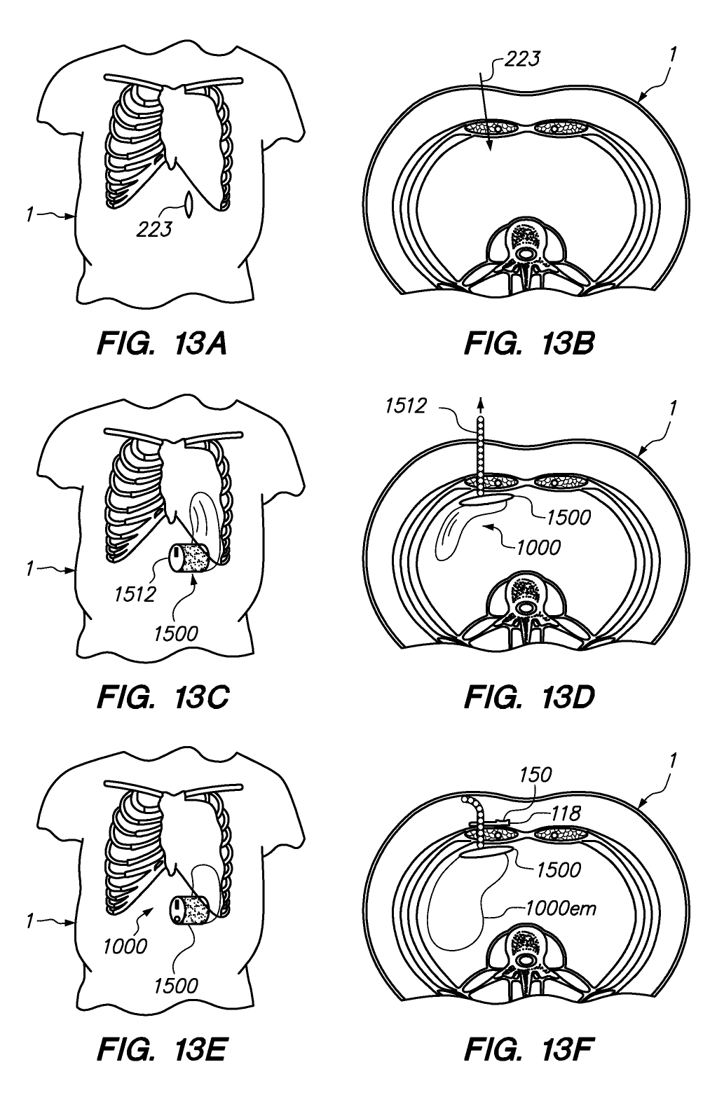

FIGS. 13A-13F illustrate a method of rapidly installing a port system in fluid communication with a device 1000 implanted in the abdominal cavity of a patient 1 according to an embodiment of the present invention. An incision 223 (see the frontal illustration of FIG. 13A and the sectional illustration of FIG. 13B) is made in the abdominal region of the patient 1 though which an opening leading into the abdominal cavity of the patient is to be established to deliver the device 1000. After forming an opening 3 from the incision 223 leading into the abdominal cavity as noted (including an incision through the muscle, see FIG. 13B and dilating it to avoid cutting an artery), the device 1000, including expandable member 1000em and attachment tab(s)/lower base 1500 are inserted through the opening 3 and into the desired position in the abdominal cavity as illustrated in FIGS. 13C-13D.

The upper base 118 (such as upper base 118 in FIG. 12C) is attached to tab (lower base) 1500 in a manner as described earlier, by pulling on ribbon 1512 and cinching upper base 118 against the fascia 127/abdominal muscle 129 to sandwich the tissues between the base 118 and base 1500 as illustrated in FIGS. 13E-13F.

For embodiments in which the base is not fixed directly to the expandable member, but rather is joined in fluid communication therewith via fill tube 12, it is not necessary to place the port directly inside the incision that was used to deliver the expandable member through. For example, the port may be placed superiorly of the incision, closer to the right costal margin. This may provide an advantage in that there is less fatty tissue near the right costal margin, so locating and accessing the port after implantation are relatively easier, such as when inflation adjustment is performed.

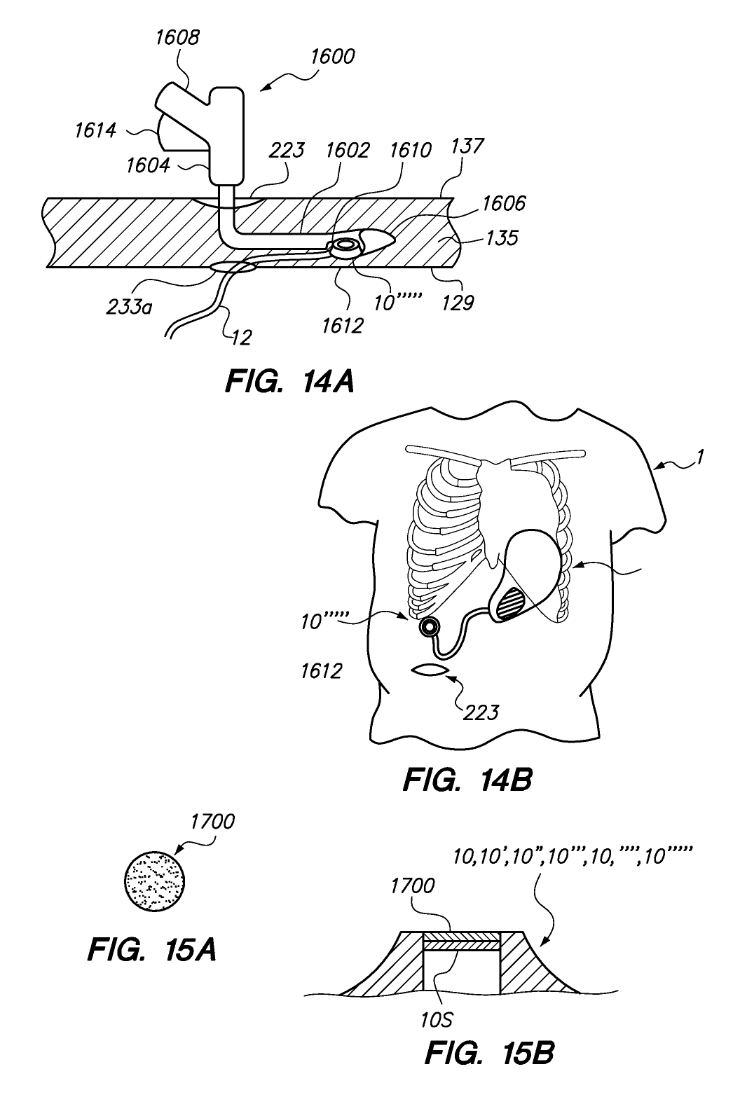

FIG. 14A shows an instrument 1600 configured and adapted to access the incision 223, tunnel subdermally from the main incision cite 223 to an adjacent location and attach the port 10′, 10″, 10″′, 10″″ to the fascia 129. Instrument 1600 includes an elongate distal shaft portion 1602 that interconnects an angled proximal shaft portion 1604 to a blunt distal tip 1606. Blunt distal tip is configured to perform blunt dissection of the fatty tissues 135 as it is driven and manipulated by an operator grasping the proximal handle 1608 extending out of the patient. The distal end portion is also configured at 1610 to engage the port device 10″″′ and drag it from the starting location at the incision 223 to the target implant location 1612 at the same time that the tunneling procedure is performed. Alternatively, the tunneling procedure can be performed first and the instrument 1600 can afterwards be retracted to engage the port device 10″″′ and then the instrument can be reinserted to deliver the port device 10″″′. In either case, trigger 1614, when actuated, drives an automatic suturing mechanism at 1610 and sutures the port 10″″′ to the target location 1612 of the fascia 129. The port device, prior to this procedure is already attached to the fill tube 12 that extends through the opening 223a into the abdominal cavity where it is in fluid communication with the expandable device.

FIG. 14B illustrates a device 1000 and port system 10″″′ having been implanted in a patient. Instrument 1600 was used in a manner as noted above to tunnel through the subcutaneous fat from the incision 223 to a target implant site 1612 superior of the incision and close to the right costal margin of the patient 1.

As noted above, there is often a substantial amount of fat between the skin 137 and the fascia 129 of a patient undergoing a procedure as described herein. At the location of the incision 223, it is not unusual to experience a thickness of up to about 7 cm of fat in the subcutaneous fat layer 135. This can make location of the port by palpation post-implantation difficult and may require the patient to go to radiology for fluoroscopic assistance in locating the port 10 so that the treatment professional can adjust the amount of inflation of the expandable member 1000 through port 10. Even a location such as 1612, it may still be advantageous to provide ways of locating the port 10,10′, 10″, 10″′,10″″,10″″′ without having to resort to fluoroscopic or x-ray visualization. However, it is important to locate the position of the port prior to inserting an inflation needle, as resort to such a blind effort can result in inadvertent sticks to the fill tubing 12 resulting in loss of efficacy of the system, which may require a subsequent surgical procedure to replace the port and/or tubing 12, not to mention unnecessary pain that may need to be borne by the patient.

FIG. 15A illustrates an optional feature that may be used in any of the port devices described herein, and which enables the port (and particularly the septum into which the fill needle is to be inserted) to be visualized by ultrasound imaging. In order to make the port more visible, particularly the septum target, a foam layer 1700 may be placed over or under the septum 10S (shown over the septum in FIG. 15B). Foam layer 1700 is preferably foamed silicone, but may be made from another biocompatible polymer foam. Foam layer 1700 may be made of open cell foam or closed cell foam or a combination of both, but is preferably closed cell to best maintain visibility under ultrasound imaging. Since air acts to reduce the transmission of ultrasound compared to the transmission through surrounding tissues, the air pockets within the foam layer 1700 are more visible than the surrounding port and tissues.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. An implantable port system, the system comprising:
a base having an upper surface configured to be positioned under and in contact with a first tissue surface in a body; and
a port housing having a lower surface configured to be positioned over a second tissue surface above the first tissue surface;
wherein said base and said port housing are separate from one another and do not contact each other at the time of positioning said base; and
wherein said base and said port are connected together upon positioning said port in contact with the second tissue surface.

2. The system of claim 1, further comprising a tissue-ingrowth encouraging layer on a top side of said base.

3. The system of claim 1, further comprising a tissue-ingrowth encouraging layer on a bottom side of said port housing.

4. The system of claim 1, wherein a bottom surface of said base comprises a tissue-ingrowth discouraging layer to discourage adhesions and tissue ingrowth.

5. The system of claim 1, further comprising means for quickly connecting said port housing to said base, said means for quickly connecting further being operable to quickly disconnect said port housing from said base.

6. The system of claim 1, wherein said base comprises a first base portion, said upper surface being provided on said first base portion, said base further comprising a second base portion have a lower surface, said lower surface of said second base portion configured to be positioned over and in contact with the second tissue surface, wherein said first and second base portions sandwich tissue including portions of the first and second tissue surfaces therebetween.

7. The system of claim 6, further comprising a quick release mechanism operable to mechanically connect said port housing to said base and further being operable to quickly disconnect said port housing from said base.

8. The system of claim 6, further comprising a ribbon interconnecting said first and second base portions, said ribbon being passable through the tissue layer.

9. The system of claim 1, wherein said base is inflatable.

10. The system of claim 1, wherein said port housing comprises a first plurality of protrusions and said base comprises a second plurality of receptacles, wherein said first plurality is a smaller number than said second plurality, whereby said port housing can be connected to said base at different relative locations through different sets of said receptacles.

11. The system of claim 10, wherein said protrusions are variably adjustable in said receptacles to vary a distance between said port housing and said base to accommodate various thicknesses of tissue layers.

12. The system of claim 6, wherein said second base portion comprises a first plurality of protrusions and said first base portion comprises a second plurality of receptacles, wherein said first plurality is a smaller number than said second plurality, whereby said second base portion can be connected to said first base portion at different relative locations through different sets of said receptacles.

13. The system of claim 12, wherein said protrusions are variably adjustable in said receptacles to vary a distance between said second base portion and said first base portion to accommodate various thicknesses of tissue layers.

14. The system of claim 1, further comprising a feature configured to facilitate location of said port housing when implanted subcutaneously.

15. The system of claim 14, wherein said feature comprises a foam layer.

16. An implantable port system, said system comprising:
base means for attachment to and contact with a tissue layer in a body; and
port means for connection to said base means after attachment of said base means to the tissue layer, wherein said port means are detachable from said base means after connecting said port means and said base means together, such that said port means are quickly releasable from and quickly attachable to said base means.

17. The system of claim 16, wherein said base means comprises a lower base portion configured and dimensioned to be attached beneath an abdominal muscle tissue layer, and an upper base portion attachable to an upper surface of the abdominal muscle tissue layer or above the upper surface of the abdominal muscle tissue layer.

18. The system of claim 16, wherein said port means is connectable to and disconnectable from said base means via a mechanical, quick-release mechanism.

19. The system of claim 16, wherein said base means comprises a lower base portion configured and dimensioned to be attached beneath the tissue layer and an upper base portion configured and dimensioned to be attached above the tissue layer, said base means further comprising mechanical connectors configured to pass through the tissue layer and connect said upper and lower base portions together.

20. The system of claim 16, wherein said base means is inflatable.

21. A port implantation system comprising:
a first elongate tube having a blunt distal end, said first elongate tube having a lumen dimensioned to receive an endoscope therein and at least a portion of said blunt distal end being transparent to allow viewing through the at least a portion thereof, said first elongate tube comprising a first mount temporarily connectable to a lower base portion of an implantable port system to mount said lower base portion thereto, at a location proximal of said blunt distal end; and
a second elongate tube having an open distal end and a lumen dimensioned to receive said first elongate tube therein, said second elongate tube comprising a second mount temporarily connectable to an upper base portion of said implantable port system to mount said upper base portion thereto, such that, when said port implantation system is assembled, said upper base portion is proximal of said lower base portion.

22. The system of claim 21, wherein said blunt distal end comprises an opening therethrough to allow passage of a tip of said endoscope.

23. The system of claim 21, wherein said blunt distal end comprises a window for viewing therethrough.

24. The system of claim 21, further comprising a third elongate tube dimensioned to be received in said second elongate tube, wherein a distal end portion of said third elongate tube comprises a housing mount temporarily connectable to a port housing of said implantable port system to mount said port housing thereto, and to advance said port housing through said second elongate tube and attach said port housing to said base after connecting said lower base portion to said upper base portion.

25. The system of claim 24, wherein said system is configured to remove said first elongate tube from said second elongate tube before inserting said third elongate tube into said second elongate tube.

26. The system of claim 21, further comprising an endoscope inserted in said first elongate tube.

27. The system of claim 21, further comprising said first elongate tube inserted in said second elongate tube;
said lower base portion mounted on said first elongate tube, proximal of said blunt distal end; and
said upper base portion mounted on said second elongate tube proximal of said lower base portion.

28. The system of claim 27, further comprising attachment members extending proximally from said lower base portion.

29. A method of implanting a port system, said method comprising:
installing at least a portion of a base of said system beneath a tissue layer in a body; and
attaching a port housing to the base at a location above the tissue layer, wherein the port housing is not in contact with the base during said installing a least a portion of the base system beneath the tissue layer.

30. The method of claim 29, wherein the base is inflatable.

31. The method of claim 29, wherein the tissue layer is fascia.

32. The method of claim 29, wherein the tissue layer comprises abdominal muscle.

33. The method of claim 29, following insertion of an expandable member having a fill tube extending proximally therefrom, wherein the base is installed over the fill tube and at least partially through an opening through which the expandable member was inserted.

34. The method of claim 29, wherein the installing includes visualizing, using an endoscope, at least placement of at least a portion of the base beneath the tissue layer.

35. The method of claim 29, wherein said installing comprises delivering the base through an elongate tube and expanding the base to have an outside diameter larger than an inside diameter of the elongate tube; and
wherein said attaching a port housing comprises manipulating attachment features to interconnect the base and the port, and cinching the port housing against one of the base and the tissue layer.

36. The method of claim 29, wherein the base comprises a balloon and the balloon is anchored beneath the tissue layer, said method further comprising:
attaching a platform above the tissue layer, to the base; and
wherein said attaching a port housing to the base comprises docking the port housing to the platform.

37. The method of claim 36, wherein the port housing is attached to the platform by a mechanism, which, in a first configuration, positions the port housing in a relatively recessed position relative to the platform, and when in a second configuration, the port housing extends upwardly from the platform.

38. The method of claim 37, further comprising pressing on and releasing pressure from the port housing to change from the first configuration to the second configuration, and from the second configuration to the first configuration, wherein the second configuration facilitates locating the port housing by touch, when use for inflation adjustment is desired.

39. The method of claim 37, wherein the mechanism is a magnetically driven mechanism, the method further comprising applying a magnetic field to the mechanism to change from the first configuration to the second configuration, and from the second configuration to the first configuration, wherein the second configuration facilitates locating the port housing by touch, when use for inflation adjustment is desired.

40. The method of claim 29, wherein said installing comprises:
inserting the base beneath the tissue layer; and
passing tethers through the base and the tissue layer, wherein the passing is in a motion from a distal to a proximal direction.

41. The method of claim 40, wherein said attaching a port housing comprises:
passing the tethers through the port housing in a distal to proximal direction;
passing the port housing over the tethers to a position in contact with the tissue layer; and
cinching the base and port housing together, thereby fixing the relative positions of the base and port housing on the tethers.

42. The method of claim 40, wherein the base is delivered beneath the tissue layer through an elongate tube.

43. The method of claim 42, further comprising removing the elongate tube after the passing tethers through the base and the tissue layer.

44. The method of claim 32, wherein said at least a portion comprises a lower base portion, and
wherein said installing comprises:
inserting a blunt tip of an instrument and the lower base portion mounted proximally of the blunt tip through an opening in the abdominal muscle;
passing connectors proximally from the lower base portion, through the tissue layer and connecting to an upper base portion of the base.

45. The method of claim 44, wherein said attaching a port housing comprises:
attaching the port housing to a tube passing through the base; and
attaching the port housing to the upper base portion.

46. The method of claim 44, wherein the blunt tip is a portion of a first elongate tube of an instrument upon which the lower base potion is mounted;
wherein the instrument further comprises a second elongate tube having a central opening through which the first elongate tube is inserted;
and wherein the upper base portion is mounted to a distal end portion of the second elongate tube, wherein said installing comprises:
passing the first and second elongate tubes through an opening in the abdominal muscle;
displacing bowel by advancing the blunt tip thereagainst;
retracting the second elongate tube from the opening in the abdominal muscle, thereby allowing the abdominal muscle and fascia to impinge upon the first elongate tube.

47. The method of claim 46, wherein said passing connectors comprises retracting the first elongate tube while substantially maintaining a position of the second elongate tube.

48. The method of claim 46, further comprising visualizing at least one step of said method, using an endoscope inserted into the first elongate tube.

49. The method of claim 29, wherein said attaching the port housing comprises screwing the port housing into the base, wherein the port housing comprises a first set of threads and the base comprises a second set of threads that mate with the first set of threads.

50. The method of claim 29, wherein said attaching the port housing comprises snapping the port housing into the base.

51. The method of claim 29, wherein said installing at least a portion of a base comprises
inserting an anvil of an instrument and the lower base portion mounted proximally of the anvil through an opening in the abdominal muscle; and
passing connectors distally through the abdominal muscle and the lower base portion, wherein the anvil prevents penetration of bowel during said passing.

52. The method of claim 51, wherein the connectors comprise T-bars.

53. The method of claim 29, wherein said at least a portion comprises a lower base portion;
wherein the lower base portion is fixed to an expandable member and the port system is configured to expand the expandable member upon injection of fluid through the port system;
wherein said installing comprises:
inserting the expandable member and lower base portion through an opening in the abdominal muscle; and
passing connectors proximally from the lower base portion, through the abdominal muscle.

54. The method of claim 53, further comprising connecting the connectors to an upper base portion of the base above the tissue layer; and
wherein said attaching a port housing comprises:
attaching the port housing to a tube passing through the base; and
attaching the port housing to the upper base portion.

55. The method of claim 53, wherein said attaching a port housing comprises:
attaching the port housing to a tube passing through the base; and
attaching the connectors to the port housing.

56. The method of claim 53, further comprising attaching the connectors to one of the port housing or an upper base portion of the base, the method further comprising connecting a ribbon between the lower base portion and one of the port housing and the upper base portion, wherein the ribbon passes through the opening and is configured to encourage tissue ingrowth therein.

57. The method of claim 29, wherein said at least a portion comprises a lower base portion;
wherein the lower base portion is fixed to an expandable member and the port system is configured to expand the expandable member upon injection of fluid through the port system;
wherein said installing comprises:
inserting the expandable member and lower base portion through an opening in the abdominal muscle; and
passing connectors distally from an upper base portion, located above the abdominal muscle into the lower base portion, thereby connecting the upper base portion to the lower base portion.

58. A method of implanting a port system into a body, said method comprising:
engaging tissue peripherally adjacent an opening in a tissue layer;
positioning a base over the opening,
positioning a port housing over the base; and
connecting the base and the port housing together and to the tissue layer while drawing the engaged tissue closer to a center of the opening.

59. The method of claim 58, wherein a tube passes through the opening and the base, said method including connecting the port housing to the tube prior to the connecting the base and the port housing together.

60. The method of claim 58, wherein the base comprises a set of outer openings and a set of inner openings, the inner openings located radially inwardly of corresponding ones of the outer openings,
wherein said engaging tissue comprises penetrating the tissue via penetrating instruments inserted through the outer openings.

61. The method of claim 60, further comprising flexible tethers, each connected at a distal end to an anchor releasably mounted to one of the penetrating instruments, the flexible tethers further being routed through respective ones of the inner opening and through openings in the port housing, wherein said connecting comprises tensioning the flexible tethers, from proximal end locations proximal of the port housing, cinching the port housing and the base down against the tissue layer and together, while the engaged tissues are also drawn radially inwardly, as directed by drawing against the inner openings.

62. The method of claim 61, wherein a tube passes through the opening and the base, said method including connecting the port housing to the tube prior to the connecting the base and the port housing together, and wherein the engaged tissues are drawn up to the tube during the connecting.

63. An implantable port system, said system comprising:
a base having a surface dimensioned to be attached over an opening through a tissue layer in a body; and
a port housing and a connector useable to connect said port housing to said base;
wherein said base comprises a set of outer openings and a set of inner openings, said inner openings located radially inwardly of corresponding ones of said outer openings.

64. The system of claim 63, further comprising a set of grooves, each one of said set of grooves extending radially between corresponding ones of said inner and outer openings.

65. The system of claim 64, further comprising penetrating instruments extendable through said outer openings and flexible tethers extendable through said inner openings, said flexible tethers being releasably mountable, at distal end portions thereof, to respective ones of distal end portions of said penetrating instruments.

66. The system of claim 65, further comprising anchors fixed to distal ends of said flexible tethers, said anchors configured to be releasably mounted to said distal end portions of said penetrating instruments.

67. An implantable port system, said system comprising:
a lower base having an upper surface configured to be positioned under an opening through tissue and in contact with a first tissue surface in a body;
an upper base having a lower surface configured to be positioned over the opening and in contact with a second tissue surface in the body; and
an injection port housing having a lower surface configured to be attached to said upper base;
wherein said lower base and said upper base each has a span dimension than a span dimension of the opening; and
wherein said lower base is compressible to a compressed configuration having a compressed span dimension less than the span dimension of the opening that allows said lower base to be passed through the opening; and
said lower base is resilient, wherein, upon passing said lower base through the opening and removing compressive forces from said lower base, said lower base resiliently returns to said span dimension greater than said span dimension of the opening, thereby preventing said lower base from passing back through said opening.

68. A method of implanting a port device, said method comprising:
accessing an incision in a patient having been previously used to deliver an implant device therethrough;
subcutaneously tunneling, through subcutaneous fat, away from the incision to a port target implant location; and
subcutaneously attaching the port device to the patient at the port target implant location.

69. An instrument for implanting a port device, said system comprising:
means for accessing an incision in a patient having been previously used to deliver an implant device therethrough;
means for subcutaneously tunneling, through subcutaneous fat, away from the incision to a port target implant location; and
means for subcutaneously attaching said port device to the patient at the port target implant location.
